(19) United States
(12) Patent Application Publication
(10) Pub. No.: US 2023/0014607 A1
(43) Pub. Date: Jan. 19, 2023

(54) METHODS AND COMPOSITIONS FOR ANALYZING NUCLEIC ACID

(71) Applicant: CLARET BIOSCIENCE, LLC, Santa Cruz, CA (US)

(72) Inventor: Richard Edward GREEN, Santa Cruz, CA (US)

(21) Appl. No.: 17/766,031

(22) PCT Filed: Oct. 8, 2020
(86) PCT No.: PCT/US20/54722

§ 371 Date: Apr. 1, 2022

Related U.S. Application Data
(60) Provisional application No. 62/913,045, filed on Oct. 9, 2019.
(60) Provisional application No. 62/938,505, filed on Nov. 21, 2019.

(51) Int. Cl. C12Q 1/6869; G16B 30/10; G16B 20/20

(52) U.S. Cl. CPC C12Q 1/6869; G16B 30/10; G16B 20/20

(57) ABSTRACT

The technology relates in part to methods and compositions for analyzing nucleic acid. In some aspects, the technology relates to methods and compositions for generating one or more genotypes.

### RELATED PATENT APPLICATIONS

This patent application is a 35 U.S.C. 371 national phase application of International Patent Cooperation Treaty (PCT) Application No. PCT/US2020/054722, filed on Oct. 8, 2020, entitled METHODS AND COMPOSITIONS FOR ANALYZING NUCLEIC ACID, naming Richard Edward GREEN as inventor, and designated by attorney docket no. CBS-2003-PC. International PCT Application No. PCT/US2020/054722 claims the benefit of U.S. provisional patent application No. 62/913,045 filed on Oct. 9, 2019, entitled METHODS AND COMPOSITIONS FOR ANALYZING NUCLEIC ACID, naming Richard Edward GREEN as inventor, and designated by attorney docket no. CBS-2003-PV. International PCT Application No. PCT/US2020/054722 also claims the benefit of U.S. provisional patent application No. 62/938,505 filed on Nov. 21, 2019, entitled METHODS AND COMPOSITIONS FOR ANALYZING NUCLEIC ACID, naming Richard Edward GREEN as inventor, and designated by attorney docket no. CBS-2003-PV2. The entire content of the foregoing applications is incorporated herein by reference, including all text, tables and drawings.

### FIELD

The technology relates in part to methods and compositions for analyzing nucleic acid. In some aspects, the technology relates to methods and compositions for generating one or more genotypes for a sample.

### BACKGROUND

Genetic information of living organisms (e.g., animals, plants and microorganisms) and other forms of replicating genetic information (e.g., viruses) is encoded in nucleic acid (i.e., deoxyribonucleic acid (DNA) or ribonucleic acid (RNA)). Genetic information is a succession of nucleotides or modified nucleotides representing the primary structure of chemical or hypothetical nucleic acids. A genotype is a part of the genetic information of a living organism, which may determine one or more of its characteristics (phenotypes). A genotype may refer to particular gene of interest, a particular mutation or marker, and/or an allele or a combination of alleles.

Existing technology, such as genotype arrays, can generate genotype data for large numbers of markers. However, certain types of samples do not have enough recoverable, high-quality nucleic acid for use with genotype arrays. This limitation is especially pronounced for certain types of forensic samples (e.g., hair, bone) where only small amounts of nucleic acid (e.g., between about 100 picograms to a few nanograms of nucleic acid) can be recovered. Provided herein is a method for accurately inferring genotypes from low-genome coverage data generated from certain types of samples (e.g., hair, bone). Genotypes are inferred through a combination of direct observation and imputation from nearby sites that are in linkage disequilibrium. Sites, i.e., known polymorphic sites, are chosen such that they will likely be correctly observed in low-coverage data. Using a method described herein, genotype files may be generated that are suitable for further analysis (e.g., genetic genealogy analysis).

### SUMMARY

Provided herein, in certain aspects, are methods for generating a genotype for a target genomic locus for a test sample, comprising a) for a test sample comprising nucleic acid, obtaining sequence reads aligned to a reference genome; b) from the sequence reads, quantifying a linked reference allele and quantifying a linked alternative allele, thereby generating allele quantifications for a linked genomic locus; c) generating a set of genotype likelihoods for a target reference allele and a target alternative allele at the target genomic locus according to 1) a probability of a genotype at the target genomic locus based, in part, on the allele quantifications in (b), and 2) a probability of a genotype at the target genomic locus based on prior probabilities of the target reference allele and the target alternative allele; and d) generating a genotype at the target genomic locus based on the set of genotype likelihoods.

Also provided herein, in certain aspects, are methods for generating a genotype for a target genomic locus for a test sample, comprising a) for a test sample comprising nucleic acid, obtaining sequence reads aligned to a reference genome; b) for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group; c) generating a haplotype pair likelihood set for the haplotype group according to i) the allele quantifications in (b), and ii) a probability of each haplotype pair; and d) generating a genotype at the target genomic locus based on the haplotype pair likelihood set in (c).

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 illustrates a genomic region showing a target site (T) and several nearby linked sites (L1, L2, L3, L4, L5, L6) within a fixed window of size w.

FIG. 2 illustrates a genomic region as in FIG. 1 with sequence reads from a forensic sample carrying allelic information at the target site and/or linked sites. Alleles are denoted 0=reference and 1=alternative alleles.

FIG. 3 shows haplotypes at any genomic region exist within the context of a phylogenetic tree, although the topology of the tree may not be known.

FIG. 4 shows alternative allele frequencies for bi-allelic markers for target sites from direct to consumer arrays.

FIG. 5 shows a general workflow for certain library preparation methods described herein.

### DETAILED DESCRIPTION

Provided herein are methods and compositions useful for analyzing nucleic acid. Also provided herein are methods and compositions for generating one or more genotypes for a sample. In certain aspects, a method herein includes generating one or more genotypes for a sample from low coverage genomic sequencing data. In certain aspects, a method herein includes generating one or more genotypes for a sample that comprises damaged, degraded and/or fragmented nucleic acid. In certain aspects, a method herein includes generating a genotype for a target genomic locus based on quantifications of a reference allele and an alternative allele for a linked genomic locus. In certain aspects, a method herein includes generating a genotype for a target genomic locus based on a haplotype analysis.

Genotyping

Provided herein are methods for generating a genotype for a target genomic locus. Also provided herein are methods for generating a plurality of genotypes for a plurality of genomic loci. In certain implementations of the methods herein, the identity of an individual and/or the identities of one or more relatives of an individual may be determined based on the plurality of genotypes generated for a plurality of genomic loci and in connection with a genealogy analysis. Certain features described below may be applicable to generating a genotype for a target genomic locus according to independent quantifications of alleles at loci that are in linkage disequilibrium with a target locus. Certain features described below may be applicable to generating a genotype for a target genomic locus using a haplotype analysis described herein.

A genotype generally refers to the genetic makeup of an organism. In particular, a genotype may refer to the alleles (e.g., variant forms of a gene and/or variant forms of a polymorphic site), that are carried by an organism. A polymorphic site may include, for example, a single nucleotide polymorphism (SNP), an insertion polymorphism, or a deletion polymorphism. Insertion and deletion polymorphisms are sometimes referred to as indels. Polymorphic sites are found throughout the genome, in coding regions and non-coding regions, and may be referred to herein as markers, target sites, and/or linked sites. Humans are diploid organisms and typically have two alleles at each genetic position, or genomic locus, with one allele inherited from each parent. When two alleles are present, the gene, trait, genomic site or polymorphism in connection with the alleles may be referred to a bi-allelic. Each pair of alleles represents the genotype of a specific gene or polymorphic site. A particular genotype is considered homozygous if it features two identical alleles and heterozygous if the two alleles differ. An allele that is more prevalent in a population (relative to the other allele at a genomic locus) may be referred to as a major allele or a reference allele. An allele that is less prevalent in a population (relative to the other allele at a genomic locus) may be referred to as a minor allele, non-reference allele, alternate allele, or an alternative allele. The process of determining a genotype is referred to as genotyping.

A method herein may comprise generating a genotype for a target genomic locus for a test sample. A target genomic locus is the locus in a genome for which a genotype is determined. A target genomic locus may be a polymorphic site in a genome. In some embodiments, a target genomic locus is a location of a single nucleotide polymorphism (SNP). In some embodiments, a target genomic locus is a location of a bi-allelic single nucleotide polymorphism (SNP). A target genomic locus may be selected based on its inclusion in one or more genealogy databases.

A genotype for a target genomic locus may be generated according to a pair of alleles. A pair of alleles may include a reference allele (i.e., major allele) and an alternative allele (i.e., minor allele, non-reference allele). A reference allele, when determined at a target genomic locus, may be referred to as a target reference allele. An alternative allele, when determined at a target genomic locus, may be referred to as a target alternative allele. Possible genotypes for a target genomic locus generally include homozygous for the target reference allele (i.e., two copies of the target reference allele), heterozygous for the target reference allele and the target alternative allele (i.e., one copy of the target reference allele and one copy of the target alternative allele), and homozygous for the target alternative allele (i.e., two copies of the target alternative allele).

In some embodiments, a genotype generated for a target genomic locus is an unphased genotype. An unphased genotype refers to a genotype lacking a designation as to which one of the pair of chromosomes (i.e., maternally inherited and paternally inherited) holds each allele. In some embodiments, a genotype generated for a target genomic locus is for a single nucleotide polymorphism (SNP). In some embodiments, a genotype generated for a target genomic locus is for a bi-allelic single nucleotide polymorphism (SNP). In some embodiments, a genotype generated for a target genomic locus is an unphased genotype for a single nucleotide polymorphism (SNP). In some embodiments, a genotype generated for a target genomic locus is an unphased genotype for a bi-allelic single nucleotide polymorphism (SNP).

In some embodiments, a method herein comprises generating a plurality of genotypes at a plurality of target genomic loci for a test sample. A plurality of genomic loci generally refers to two or more genomic loci. In some embodiments, a plurality of genomic loci comprises about 1,000 or more genomic loci. In some embodiments, a plurality of genomic loci comprises about 10,000 or more genomic loci. In some embodiments, a plurality of genomic loci comprises about 100,000 or more genomic loci. In some embodiments, a plurality of genomic loci comprises about 200,000 or more genomic loci. In some embodiments, a plurality of genomic loci comprises about 300,000 or more genomic loci. In some embodiments, a plurality of genomic loci comprises about 400,000 or more genomic loci. In some embodiments, a plurality of genomic loci comprises about 500,000 or more genomic loci. In some embodiments, a plurality of genomic loci comprises about 600,000 or more genomic loci. In some embodiments, a plurality of genomic loci comprises about 700,000 or more genomic loci. In some embodiments, a plurality of genomic loci comprises about 800,000 or more genomic loci. In some embodiments, a plurality of genomic loci comprises about 900,000 or more genomic loci. In some embodiments, a plurality of genomic loci comprises about 1,000,000 or more genomic loci.

In some embodiments, a method herein comprises filtering one or more target genomic loci. Filtering one or more target genomic loci refers to removing one or more target genomic loci from a genotyping analysis herein. In some embodiments, one or more target genomic loci are filtered by removing genomic loci that are within a certain proximity of an insertion polymorphism or a deletion polymorphism. In some embodiments, one or more target genomic loci are filtered by removing genomic loci that are within 1 to 10 bases of an insertion polymorphism or a deletion polymorphism. For example, a target genomic locus may be removed from a genotyping analysis herein if the target genomic locus is within 1 base, 2, bases, 3 bases, 4 bases, 5 bases, 6 bases, 7 bases, 8 bases, 9 bases, or 10 bases of an insertion polymorphism or a deletion polymorphism. In some embodiments, a target genomic locus is removed from a genotyping analysis herein if the target genomic locus is within 4 bases of an insertion polymorphism or a deletion polymorphism. A genomic target site filtered according to criteria described above may be referred to as a blacklisted site.

In some embodiments, alleles are analyzed at a linked genomic locus. A linked genomic locus generally refers to a genomic locus located within a certain proximity of a target genomic locus. In some embodiments, a linked genomic locus refers to a genomic locus located within about 1 kilobase (kb) to about 20 kb (upstream or downstream) of a target genomic locus. For example, a linked genomic locus may be within about 1 kb, about 2 kb, about 3 kb, about 4 kb, about 5 kb, about 6 kb, about 7 kb, about 8 kb, about 9 kb, about 10 kb, about 11 kb, about 12 kb, about 13 kb, about 14 kb, about 15 kb, about 16 kb, about 17 kb, about 18 kb, about 19 kb, or about 20 kb of a target genomic locus. In some embodiments, a linked genomic locus is a genomic locus located within about 10 kb upstream or within 10 kb downstream of a target genomic locus. A linked genomic locus may be a polymorphic site in a genome. In some embodiments, a linked genomic locus is a location of a single nucleotide polymorphism (SNP). In some embodiments, a linked genomic locus is a location of a bi-allelic single nucleotide polymorphism (SNP). A linked genomic locus may be selected based on its inclusion in one or more databases. For example, linked genomic loci may be selected according to one or more human genome sequencing projects (e.g., the 1000 Genomes project). In some embodiments, genotypes and genotype likelihoods are not determined for linked genomic loci. In such instances, genotypes and genotype likelihoods are generated for one or more target genomic loci without generating genotypes or genotype likelihoods for one or more linked genomic loci. In some embodiments, genotypes and genotype likelihoods are determined for linked genomic loci. In such instances, genotypes and genotype likelihoods are generated for one or more target genomic loci, based, in part, on genotypes or genotype likelihoods generated for one or more linked genomic loci.

In some embodiments, alleles are analyzed at a plurality of linked genomic loci. In some embodiments, alleles are analyzed at a plurality of linked genomic loci for each target genomic locus. A plurality of linked genomic loci may comprise about 10 linked genomic loci to about 1000 linked genomic loci (e.g., for each target genomic locus). For example, a plurality of linked genomic loci may comprise about 10 linked genomic loci, about 20 linked genomic loci, about 30 linked genomic loci, about 40 linked genomic loci, about 50 linked genomic loci, about 60 linked genomic loci, about 70 linked genomic loci, about 80 linked genomic loci, about 90 linked genomic loci, about 100 linked genomic loci, about 200 linked genomic loci, about 300 linked genomic loci, about 400 linked genomic loci, about 500 linked genomic loci, about 600 linked genomic loci, about 700 linked genomic loci, about 800 linked genomic loci, about 900 linked genomic loci, or about 1000 linked genomic loci. A plurality of linked genomic loci may comprise about 5 linked genomic loci to about 50 linked genomic loci (e.g., for each target genomic locus). For example, a plurality of linked genomic loci may comprise about 5 linked genomic loci, about 10 linked genomic loci, about 15 linked genomic loci, about 20 linked genomic loci, about 25 linked genomic loci, about 30 linked genomic loci, about 35 linked genomic loci, about 40 linked genomic loci, about 45 linked genomic loci, or about 50 linked genomic loci.

In some embodiments, allele quantifications are generated. In some embodiments, allele quantifications are generated for alleles at a linked genomic locus. For example, a method herein may comprise quantifying a linked reference allele (major allele at a linked genomic locus) and quantifying a linked alternative allele (minor allele at a linked genomic locus). In some embodiments, allele quantifications are generated for alleles at a target genomic locus. For example, a method herein may comprise quantifying a target reference allele and quantifying a target alternative allele. Each allele quantification may be generated according to the amount of sequence reads, or an adjusted amount of sequence reads, carrying a particular allele at a genomic locus. For example, an allele quantification may be generated according to the amount of sequence reads carrying a reference allele at a linked genomic locus. In some embodiments, an allele quantification is generated according to the amount of sequence reads carrying an alternative allele at a linked genomic locus. In some embodiments, an allele quantification is generated according to the amount of sequence reads carrying a reference allele at a target genomic locus. In some embodiments, an allele quantification is generated according to the amount of sequence reads carrying an alternative allele at a target genomic locus. The amount of sequence reads for an allele quantification may be adjusted, for example, according to a measure of sequencing error. In some embodiments, a measure of sequencing error is a fixed error rate (e.g., a fixed error rate associated with a particular sequencing platform and/or sequencing library preparation method). In some embodiments, a measure of sequencing error is an error associated with a sequencing run, a test sample, or group of test samples. In some embodiments, a measure of sequencing error is an error associated with a particular genomic locus or region.

In some embodiments, a method herein comprises quantifying a plurality of linked reference alleles and quantifying a plurality of linked alternative alleles, thereby generating a plurality of allele quantifications for a plurality of linked genomic loci. In some embodiments, a plurality of linked reference alleles and a plurality of linked alternative alleles are quantified at a plurality of linked genomic loci for each target genomic locus. Accordingly, generating a genotype call at a target genomic locus may be based on allele quantifications at a plurality of linked genomic loci. Furthermore, generating a plurality of genotypes at a plurality of linked genomic loci may be based on allele quantifications at multiple pluralities of linked genomic loci (i.e., each genotype call is based on allele quantifications at its own set of linked genomic loci).

An allele quantification can be determined by a suitable method, operation or mathematical process. An allele quantification sometimes is the direct sum of all sequence reads carrying a particular allele (e.g., reference allele, alternative allele) at a genomic locus (e.g., a liked genomic locus, a target genomic locus). An allele quantification may be expressed as a ratio (e.g., a ratio of a quantification for a particular allele to a quantification for a different allele or all alleles).

In some embodiments, an allele quantification is derived from raw sequence reads and/or filtered sequence reads. In certain embodiments, an allele quantification is an average, mean or sum of sequence reads carrying a particular allele (e.g., reference allele, alternative allele) at a genomic locus (e.g., a liked genomic locus, a target genomic locus). In some embodiments, an allele quantification is associated with an uncertainty value. An allele quantification sometimes is adjusted. An allele quantification may be adjusted according to sequence reads that have been weighted, removed, filtered, normalized, adjusted, averaged, derived as a mean, derived as a median, added, or combination thereof.

In some embodiments, a method herein comprises filtering one or more sequence reads. Filtering one or more sequence reads refers to removing one or more sequence reads from a genotyping analysis herein. In some embodiments, one or more sequence reads are filtered by removing sequence reads that align to a genomic position within a certain proximity of an insertion polymorphism or a deletion polymorphism. In some embodiments, one or more sequence reads are filtered by removing sequence reads that align to a genomic position within 1 to 10 bases of an insertion polymorphism or a deletion polymorphism. For example, a sequence read may be removed from a genotyping analysis herein if the sequence read aligns to a genomic position within 1 base, 2, bases, 3 bases, 4 bases, 5 bases, 6 bases, 7 bases, 8 bases, 9 bases, or 10 bases of an insertion polymorphism or a deletion polymorphism. In some embodiments, a sequence read is removed from a genotyping analysis herein if the sequence read aligns to a genomic position within 4 bases of an insertion polymorphism or a deletion polymorphism.

In some embodiments, a method herein comprises filtering sequence reads according to mapping/alignment parameters and/or quality score. For example, sequence reads that do not map well and/or do not have a suitable alignment score may be removed from an analysis herein. Reads that may be filtered out include, for example, discordant reads, ambiguous reads, off-target reads, reads having one or more undetermined base calls, and reads having a low quality sequences and/or base quality scores. Low quality sequences may be identified according to base quality scores for one or more nucleotide positions in a sequence. A base quality score, or quality score, is a prediction of the probability of an error in base calling. Quality scores may be generated according to one or more sets of quality predictor values, and can depend on certain characteristics of the sequencing platform used for generating sequence reads. Generally, a high quality score indicates a base call is more reliable and less likely is an incorrect base call. In some embodiments, individual bases within sequence reads are filtered. For example, individual bases that do not have a suitable base quality score may be removed from an analysis herein.

In some embodiments, a method herein comprises filtering one or more allele quantifications. Filtering one or more allele quantifications refers to removing one or more allele quantifications from a genotyping analysis herein. In some embodiments, one or more allele quantifications are filtered by removing allele quantifications derived from sequence reads that align to a genomic position within a certain proximity of an insertion polymorphism or a deletion polymorphism. In some embodiments, one or more allele quantifications are filtered by removing allele quantifications derived from sequence reads that align to a genomic position within 1 to 10 bases of an insertion polymorphism or a deletion polymorphism. For example, an allele quantification may be removed from a genotyping analysis herein if the allele quantification is derived from sequence reads aligned to a genomic position within 1 base, 2, bases, 3 bases, 4 bases, 5 bases, 6 bases, 7 bases, 8 bases, 9 bases, or 10 bases of an insertion polymorphism or a deletion polymorphism. In some embodiments, an allele quantification is removed from a genotyping analysis herein if the allele quantification is derived from sequence reads aligned to a genomic position within 4 bases of an insertion polymorphism or a deletion polymorphism.

In some embodiments, a method herein comprises determining one or more genotype likelihoods for a target genomic locus. In some embodiments, a method herein comprises determining one or more genotype likelihoods for a target reference allele and a target alternative allele at a target genomic locus. In some embodiments, a method herein comprises determining a set of genotype likelihoods for a target reference allele and a target alternative allele at a target genomic locus. A set of genotype likelihoods may comprise one or more likelihoods for genotypes chosen from homozygous for the target reference allele, heterozygous for the target reference allele and the target alternative allele, and homozygous for the target alternative allele. In some embodiments, a set of genotype likelihoods comprises likelihoods for a homozygous for the target reference allele genotype, a heterozygous for the target reference allele and the target alternative allele genotype, and a homozygous for the target alternative allele genotype.

In some embodiments, a method herein comprises generating a genotype likelihood for a target reference allele and a target alternative allele at a target genomic locus according to one or more probabilities of a genotype at the target genomic locus. In some embodiments, a method herein comprises generating a set of genotype likelihoods for a target reference allele and a target alternative allele at a target genomic locus according to probabilities for each genotype (i.e., homozygous reference, heterozygous reference and alternative, and homozygous alternative) at the target genomic locus. A probability of a genotype at a target genomic locus may be based, in part, on one or more of allele frequency, haplotype frequency, genotype frequency, allele quantifications, and prior probabilities.

In some embodiments, a method herein comprises generating a genotype likelihood for a target reference allele and a target alternative allele at a target genomic locus according to one or more probabilities of observing certain data obtained for a test sample (e.g., allele quantifications obtained for a test sample) given a particular genotype at a target genomic locus. The phrase “given a particular genotype at a target genomic locus” refers to an assumption of a particular genotype at a target genomic locus. The phrase “given a particular genotype at a target genomic locus” may be used interchangeably with “for a particular assumed genotype at a target genomic locus.” For example, a method herein may comprise using observed allele quantifications at one or more linked genomic loci to query 1) how likely such observed allele quantifications would be if the genotype at the target genomic locus was homozygous reference; 2) how likely such observed allele quantifications would be if the genotype at the target genomic locus was heterozygous; and/or 3) how likely such observed allele quantifications would be if the genotype at the target genomic locus was homozygous alternative.

In some embodiments, a probability of a genotype at a target genomic locus is based, in part, on data obtained for a test sample (e.g., allele quantifications obtained for a test sample). For example, a probability of a genotype at a target genomic locus may be based, in part, on allele quantifications for a linked reference allele and a linked alternative allele. In some embodiments, a probability of a genotype at a target genomic locus may be further based, in part, on allele quantifications for a target reference allele and a target alternative allele.

In some embodiments, a probability of a genotype at a target genomic locus is generated according to a probability of observing a particular allele (e.g., reference or alternative) at a linked genomic locus, given a particular allele (e.g., reference or alternative) or genotype at a target genomic locus. The phrase “given a particular allele (e.g., reference or alternative) or genotype at a target genomic locus” refers to an assumption of a particular allele or genotype at a target genomic locus. The phrase “given a particular allele (e.g., reference or alternative) or genotype at a target genomic locus” may be used interchangeably with “for a particular assumed allele (e.g., reference or alternative) or assumed genotype at a target genomic locus.” In some embodiments, a probability of a genotype at a target genomic locus is generated according to a probability of observing a linked reference allele at a linked genomic locus, given a target reference allele at a target genomic locus. In some embodiments, a probability of a genotype at a target genomic locus is generated according to a probability of observing a linked reference allele at a linked genomic locus, given a target alternative allele at a target genomic locus. In some embodiments, a probability of a genotype at a target genomic locus is generated according to a probability of observing a linked reference allele at a linked genomic locus, given a target reference allele at a target genomic locus, and a probability of observing a linked reference allele at a linked genomic locus, given a target alternative allele at a target genomic locus. In some embodiments, the probability of a genotype that is homozygous for a target reference allele is generated according to a probability of observing a linked reference allele at a linked genomic locus, given a target reference allele at a target genomic locus. In some embodiments, the probability of a genotype that is heterozygous for a target reference allele and a target alternative allele is generated according to a probability of observing a linked reference allele at a linked genomic locus, given a target reference allele at a target genomic locus, and a probability of observing a linked reference allele at a linked genomic locus, given a target alternative allele at a target genomic locus. In some embodiments, the probability of a genotype that is homozygous for a target alternative allele is generated according to a probability of observing the linked reference allele at the linked genomic locus, given a target alternative allele at the target genomic locus.

In some embodiments, a probability of a genotype at a target genomic locus is generated according to a probability of observing a linked reference allele at a linked genomic locus, given a target reference genotype at a target genomic locus. In some embodiments, a probability of a genotype at a target genomic locus is generated according to a probability of observing a linked reference allele at the linked genomic locus, given a target alternative genotype at a target genomic locus. In some embodiments, a probability of a genotype at a target genomic locus is generated according to a probability of observing a linked reference allele at a linked genomic locus, given a target reference genotype at a target genomic locus, and a probability of observing a linked reference allele at a linked genomic locus, given a target alternative genotype at a target genomic locus. In some embodiments, the probability of a genotype that is homozygous for a target reference allele is generated according to a probability of observing a linked reference allele at a linked genomic locus, given a target reference genotype (e.g., homozygous reference) at a target genomic locus. In some embodiments, the probability of a genotype that is heterozygous for a target reference allele and a target alternative allele is generated according to a probability of observing a linked reference allele at a linked genomic locus, given a target reference genotype at a target genomic locus, and a probability of observing a linked reference allele at a linked genomic locus, given a target alternative genotype at a target genomic locus. In some embodiments, the probability of a genotype that is heterozygous for a target reference allele and a target alternative allele is generated according to a probability of observing a linked reference allele at a linked genomic locus, given a heterozygous genotype at a target genomic locus. In some embodiments, the probability of a genotype that is homozygous for a target alternative allele is generated according to a probability of observing the linked reference allele at the linked genomic locus, given a target alternative genotype (e.g., homozygous alternative) at the target genomic locus.

In some embodiments, a probability of observing a linked reference allele at a linked genomic locus, given a particular target allele or genotype at a target genomic locus, is based, in part, on a measure of linkage disequilibrium for a linked reference allele and a target reference allele. Linkage disequilibrium refers to a non-random association of alleles at two or more loci (e.g., in a population). In some embodiments, a measure of disequilibrium is based on a haplotype frequency (e.g., a haplotype frequency in a population for a linked reference allele and a particular target allele (e.g., reference or alternative)).

In some embodiments, a probability of observing a linked reference allele at a linked genomic locus, given a particular target allele or genotype at a target genomic locus, is combined with an allele quantification (e.g., an allele quantification of linked reference alleles, an allele quantification of linked alternative alleles). A probability may be combined with an allele quantification by applying a mathematical manipulation. A mathematical manipulation may include, for example, multiplication, division, addition, subtraction, integration, symbolic computation, algebraic computation, algorithm, trigonometric or geometric function, transformation, and a combination thereof. Examples of a probability of observing a linked reference allele at a linked genomic locus, given a particular target allele or genotype at a target genomic locus, combined with an allele quantification are provided in equations (1) and (2) herein.

In some embodiments, a probability of observing a linked reference allele at a linked genomic locus, given a particular target allele or genotype at a target genomic locus, may be adjusted. In some embodiments, a probability is adjusted according to a measure of sequencing error. In some embodiments, a measure of sequencing error is a fixed error rate (e.g., a fixed error rate associated with a particular sequencing platform and/or sequencing library preparation method). In some embodiments, a measure of sequencing error is an error associated with a sequencing run, a test sample, or group of test samples. In some embodiments, a measure of sequencing error is an error associated with a particular genomic locus or region.

In some embodiments, a probability of a genotype at a target genomic locus is based, in part, on one or more prior probabilities. Prior probabilities may be based on certain frequencies in a population (e.g., allele frequencies, genotype frequencies, haplotype frequencies). In some embodiments, a probability of a genotype at a target genomic locus is based on prior probabilities of the target reference allele and the target alternative allele. Prior probabilities may be based, in part, on haplotype frequencies (e.g., haplotype frequencies in a population). For example, prior probabilities may be based on haplotype frequencies for one or more or all of (i) a target reference allele and a linked reference allele, (ii) a target reference allele and a linked alternative allele, (iii) a target alternative allele and a linked reference allele, and (iv) a target alternative allele and a linked alternative allele.

In some embodiments, a likelihood (L) for a homozygous target reference allele genotype (T00) is generated according to a process derived from equation (1):

$$L ( T 00 ) = P ( D \vert T 00 ) \times P ( T 00 ) = {\prod }_{{L}_{i}} ( \begin{matrix}{L}_{i} 0 + {L}_{i} 1 \\ {L}_{i} 0\end{matrix} ) \times {PL}_{i} {0}^{{L}_{i} 0} \times {( 1 - P {L}_{i} 0 )}^{{L}_{i} 1} \times {( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} )}^{2} \qquad ( 1 )$$

where
L(T00) is a likelihood of genotype 00 at target genomic locus T,
Lᵢ0 is an allele quantification of linked reference alleles observed at linked genomic locus Lᵢ (where ᵢ refers to the linked genomic locus being analyzed),
Lᵢ1 is an allele quantification of linked alternative alleles observed at linked genomic locus Lᵢ,
PLᵢ0 is a probability of observing a linked reference allele at linked genomic locus Lᵢ, given allele T0, and
T0Lᵢ0, T0Lᵢ1, T1Lᵢ0 and T1Lᵢ1 are haplotype frequencies for (i) a target reference allele and a linked reference allele, (ii) a target reference allele and a linked alternative allele, (iii) a target alternative allele and a linked reference allele, and (iv) a target alternative allele and a linked alternative allele.

In some embodiments, a likelihood (L) for a homozygous target alternative allele genotype (T11) is generated according to a process derived from equation (2):

$$L ( T 11 ) = P ( D \vert T 11 ) \times P ( T 11 ) = {\prod }_{{L}_{i}} ( \begin{matrix}{L}_{i} 0 + {L}_{i} 1 \\ {L}_{i} 0\end{matrix} ) \times {PL}_{i} {0}^{{L}_{i} 0} \times {( 1 - P {L}_{i} 0 )}^{{L}_{i} 1} \times {( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} )}^{2} \qquad ( 2 )$$

where:
L(T11) is a likelihood of genotype 11 at target genomic locus T
Lᵢ0 is an allele quantification of linked reference alleles observed at linked genomic locus Lᵢ
Lᵢ1 is an allele quantification of linked alternative alleles observed at linked genomic locus Lᵢ
PLᵢ0 is a probability of observing a linked reference allele at linked genomic locus Lᵢ, given allele T1, and
T0Lᵢ0, T0Lᵢ1, T1Lᵢ0 and T1Lᵢ1 are haplotype frequencies for (i) a target reference allele and a linked reference allele, (ii) a target reference allele and a linked alternative allele, (iii) a target alternative allele and a linked reference allele, and (iv) a target alternative allele and a linked alternative allele.

In some embodiments, a likelihood (L) for a heterozygous target reference allele and target alternative allele genotype (T01) is generated according to a process derived from equation (1) and equation (2). For example, a likelihood (L) for a heterozygous target reference allele and target alternative allele genotype (T01) is generated according to a process derived from equation (3):

$$L ( T 01 ) = P ( D \vert T 01 ) \times P ( T 01 ) = \prod {L}_{i} ( \begin{matrix}{L}_{i} 0 + {L}_{i} 1 \\ {L}_{i} 0\end{matrix} ) \times P {L}_{i} {0}^{{L}_{i} 0} \times {( 1 - P {L}_{i} 0 )}^{{L}_{i} 0} \times ( 2 \times ( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} ) \times ( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} ) ) \\ where : \\ {PL}_{i} 0 = ( 0.5 \times \frac{T 0 {L}_{i} 0}{T 0 {L}_{i} 0 + T 0 {L}_{i} 1} ) + ( 0.5 \times \frac{T 1 {L}_{i} 0}{T 1 {L}_{i} 0 + T 1 {L}_{i} 1} ) \qquad ( 3 )$$

In some embodiments, a plurality of genotype likelihood sets for a target genomic locus is generated. In some embodiments, a plurality of genotype likelihood sets for a target genomic locus is generated according to a plurality of allele quantifications for a plurality of linked genomic loci. For example, for a target genomic locus having 10 linked genomic loci, 10 genotype likelihood sets may be generated. In another example, for a target genomic locus having 100 linked genomic loci, 100 genotype likelihood sets may be generated. In some embodiments, a genotype at a target genomic locus is generated based on a plurality of genotype likelihood sets.

A genotype at a target genomic locus may be generated based on a set of genotype likelihoods. As described above each set of genotype likelihoods is generated according to allele quantifications and probabilities for a linked genomic locus, and each set may contain a likelihood for each genotype possibility at the target genomic locus: homozygous reference, heterozygous reference/alternative, and homozygous alternative. In instances where three genotype likelihoods are generated for a target genomic locus, based on a first linked genomic site, the most likely genotype is selected. The likelihood of the most likely genotype may be compared to the likelihood of the second most likely genotype to generate a likelihood ratio. Genotype calls may be filtered according to this ratio, calling only genotypes with a high likelihood ratio and/or a ratio above a particular threshold or cutoff value. For example, genotypes calls in which the most likely call is at least 10 times, at least 100 times, at least 1000 times, or at least 10,000 times more likely than the second most likely call may be reported. Thus, a high likelihood ratio and/or a ratio above a particular threshold or cutoff value generally refers to a ratio value of about 10 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, 1000 or more, 2000 or more, 3000 or more, 4000 or more, 5000 or more, 6000 or more, 7000 or more, 8000 or more, 9000 or more, or 10,000 or more. In certain instances, when a likelihood ratio is below a threshold or cutoff, a partial genotype may be generated by calling one of the alleles at the target genomic locus.

In some embodiments, each genotype for a target genomic locus in a plurality of genotypes for a plurality of genomic loci is generated independently from the other genotypes in the plurality of genotypes. Accordingly, a genotype generated at a first locus has no bearing on a genotype generated at a second locus, even if the first locus and the second locus are within a certain proximity to each other (e.g., are considered linked target loci). Thus, if a genotype generated at a first locus is an erroneous genotype, the genotype determined at the second locus is not any more likely to be erroneous. Methods herein typically generate genotypes at target genomic loci without generating a haplotype for two or more target genomic loci. A haplotype generally refers to a group of alleles that are inherited together from one parent. In some contexts, a haplotype refers to a collection of specific alleles in a cluster of tightly linked genes on a chromosome that are likely to be inherited together. In some contexts, a set of linked single nucleotide polymorphism (SNP) alleles that are associated statistically. Certain existing genotyping approaches use a few alleles of a specific haplotype sequence to identify other polymorphic sites that are nearby on the chromosome. For example, certain existing genotyping approaches generate genotypes at all sites (all target sites and all linked sites) in a panel, and attempt to learn which two haplotypes are present. Using this approach, when a haplotype is called incorrectly, correlated errors are made, outputting alleles of the wrong haplotype. In contrast, the genotyping approach described herein considers every target genomic locus (i.e., every target genomic locus along with its linked genomic locus) independently. Thus, errors are independent and uncorrelated. Generally, genotype calls are made at target genomic loci (and not linked genomic loci). Linked genomic loci generally are used independently for target sites. In certain instances, a linked genomic locus could be a linked genomic locus for two or more target genomic loci. In such instance, the linked genomic loci is used independently for each target site.

Because each sequence read is independent data from other sequence reads, genotype likelihoods calculated from data at each linked site generally are treated as independent observations. In some embodiments, a composite genotype likelihood is generated according to a plurality of linked genomic loci likelihoods for each target genomic locus genotype possibility. In some embodiments, a composite genotype likelihood is generated by multiplying all linked genomic loci likelihoods for each target genomic locus genotype possibility. In some embodiments, results may be filtered according to a level of linkage disequilibrium and/or observed coverage. In some embodiments, a genotype likelihood ratio is generated according to a comparison of composite likelihoods of each target genomic locus genotype (homozygous reference, heterozygous reference/alternative, and homozygous alternative). This ratio may be used to filter results in the context of one or more considerations of probability, non-limiting examples of which include, sensitivity, specificity, standard deviation, median absolute deviation (MAD), measure of certainty, measure of confidence, measure of certainty or confidence that a value obtained for a genotype likelihood ratio is inside or outside a particular range of values, measure of uncertainty, measure of uncertainty that a value obtained for a genotype likelihood ratio is inside or outside a particular range of values, coefficient of variation (CV), confidence level, confidence interval (e.g., about 95% confidence interval), standard score (e.g., z-score), chi value, phi value, result of a t-test, p-value, ploidy value, fitted minority species fraction, area ratio, median level, the like or combination thereof. For example, a genotype likelihood ratio may be used to filter results at any desired confidence level. In some embodiments, suitable genotype likelihood ratios for genetic genealogy range from about 10 to 10,000.

Using a genotyping method described herein, genotype calls may be made for a majority of target genomic loci. In some embodiments, genotype calls are made for at least about 75% of the target genomic loci. For example, genotype calls may be made for at least 80%, at least 85%, at least 90%, at least 91%, at least 92%, at least 93%, at least 94%, at least 95%, at least 96%, at least 97%, at least 98%, at least 99%, or 100% of target genomic loci. In some embodiments, genotype calls are made for about 92% of the target genomic loci.

In some embodiments, a method herein comprises identifying a subject based on a plurality of genotypes generated for a test sample. In some embodiments, genotypes generated according to a method provided herein are entered into a file format suitable for downstream analysis (e.g., uploaded to a genetic genealogy service). In some embodiments, a subject from which the sample was derived is identified according to a downstream analysis (e.g., analysis performed by a genetic genealogy service or analysis performed using a database connected to a genetic genealogy service). In some embodiments, one or more relatives of a subject from which the sample was derived is/are identified according to a downstream analysis (e.g., analysis performed by a genetic genealogy service or analysis performed using a database connected to a genetic genealogy service). Generally, accurate genotype calls at a large number of target genomic loci are required for a positive identification of a subject or a relative of a subject. For example, using certain genealogy platforms, at least about 100,000, 200,000, 300,000, 400,000, 500,000, 600,000, 700,000, 800,000, 900,000 or 1,000,000 accurate genotype calls are required. In some embodiments, at least 500,000 accurate genotype calls are required for a positive identification of a subject or a relative of a subject. In some embodiments, at least 600,000 accurate genotype calls are required for a positive identification of a subject or a relative of a subject.

Genotyping Using a Haplotype Analysis

Provided herein are methods for generating a genotype for a target genomic locus according to a haplotype analysis. A haplotype generally refers to a group of alleles that are inherited together (i.e., on the same chromosome or chromosome section) from a single parent. In some embodiments, a method herein comprises analyzing a haplotype group. A haplotype group herein generally refers a section of a genome comprising a target genomic locus and a plurality of linked genomic loci. A haplotype group may be referred to herein as a haplotype set, a haplotype panel, or a haplotype description. A haplotype group may be described as a matrix where the rows are unique haplotypes and the columns are the genomic loci in the haplotypes, as described in Example 2.

The linked genomic loci that make up a haplotype group may be selected according to one or more criteria described herein. For example, a haplotype group may comprise linked genomic loci in linkage disequilibrium with a target genomic locus. A haplotype group may comprise linked genomic loci generally present in nucleic acid recovered from a particular type of test sample (e.g., hair DNA, damaged or degraded DNA). A haplotype group may comprise linked genomic loci having suitable mapping characteristics (e.g., loci that avoid repetitive regions, loci that avoid insertion-deletion polymorphisms, and loci that avoid other genome features that may disrupt accurate mapping). Genomic loci in a haplotype group may be adequately spaced from one another (e.g., spaced such that a single sequencing read generally does not comprise multiple loci, thus avoiding over counting data from single reads). In some embodiments, each locus in the plurality of linked genomic loci in the haplotype group is at least about 1 base away to at least about 250 bases away from other loci in the haplotype group. For example, each locus in the plurality of linked genomic loci in the haplotype group may be at least about 10 bases away from other loci in the haplotype group, at least about 20 bases away from other loci in the haplotype group, at least about 30 bases away from other loci in the haplotype group, at least about 40 bases away from other loci in the haplotype group, at least about 50 bases away from other loci in the haplotype group, at least about 60 bases away from other loci in the haplotype group, at least about 70 bases away from other loci in the haplotype group, at least about 80 bases away from other loci in the haplotype group, at least about 90 bases away from other loci in the haplotype group, at least about 100 bases away from other loci in the haplotype group, at least about 150 bases away from other loci in the haplotype group, at least about 200 bases away from other loci in the haplotype group, or at least about 250 bases away from other loci in the haplotype group. In some embodiments, each locus in the plurality of linked genomic loci in the haplotype group is at least about 70 bases away from other loci in the haplotype group.

In some embodiments, allele quantifications are generated for a haplotype group. Generating allele quantifications is described above. In some embodiments, a method herein comprises quantifying linked alleles in a haplotype group. In some embodiments, a method herein comprises quantifying a linked reference allele and quantifying a linked alternative allele for each linked genomic locus in a haplotype group. In some embodiments, a method herein comprises quantifying a target allele in a haplotype group. In some embodiments, a method herein comprises quantifying a target reference allele and quantifying a target alternative allele for a target genomic locus in a haplotype group. In some embodiments, allele quantifications are generated for a plurality of haplotype groups. In some embodiments, a method herein comprises quantifying linked alleles in a plurality of haplotype groups. In some embodiments, a method herein comprises quantifying a linked reference allele and quantifying a linked alternative allele for each linked genomic locus in each haplotype group, thereby generating allele quantifications for each linked genomic locus for each group in the plurality of haplotype groups.

In some embodiments, a likelihood for one or more haplotype pairs is generated. A haplotype pair refers to, for a diploid organism, two haplotypes (two haplotype species) from the same haplotype group, where the first haplotype is on one chromosome (inherited from one parent) and the second haplotype is on the homologous chromosome (inherited from the other parent). A haplotype pair may comprise any two possible haplotype species for a haplotype group. A haplotype pair may comprise two identical haplotype species (e.g., AA from the haplotypes in Table 2 in Example 2) or may comprise two different haplotype species (e.g., AB from the haplotypes in Table 2 in Example 2). A likelihood for a haplotype pair generally refers to a measure of how likely a test sample (e.g., from a test subject) has particular haplotype pair given the observed data (e.g., allele quantifications). In some embodiments, a likelihood for each possible haplotype pair is generated. A haplotype pair likelihood may be based, in part, on one or more of allele frequency, haplotype frequency, genotype frequency, allele quantifications, and prior probabilities.

In some embodiments, a haplotype pair likelihood set is generated for a haplotype group. In some embodiments, a haplotype pair likelihood set is generated according to allele quantifications for each linked genomic locus for a haplotype group. A haplotype pair likelihood set generally refers to a collection of likelihoods generated for a plurality of haplotype pair possibilities (e.g., where the set comprises a separate likelihood for each haplotype pair possibility in Table 2: AA, AB, BB, BC, CC, etc.). In some embodiments, a plurality of haplotype pair likelihood sets are generated for a plurality of haplotype groups. In some embodiments, a plurality of haplotype pair likelihood sets are generated according to allele quantifications for each linked genomic locus for each group in a plurality of haplotype groups.

A haplotype pair likelihood set may be generated according to any suitable statistical process or model. In some embodiments, a haplotype pair likelihood set is generated according to an evidential probability. In some embodiments, a haplotype pair likelihood set is generated according to a Bayesian probability. A Bayesian probability generally refers to an interpretation of the concept of probability, where probability is interpreted as reasonable expectation. A Bayesian interpretation of probability may be considered an extension of propositional logic that enables reasoning with hypotheses, with propositions whose truth or falsity is unknown, where a probability is assigned to a hypothesis. To evaluate the probability of a hypothesis, a prior probability is specified, which may be updated to a posterior probability in view of relevant data.

In some embodiments, a method herein comprises generating a haplotype pair likelihood set for a haplotype group according to i) allele quantifications and ii) a probability of each haplotype pair. In some embodiments, a haplotype pair likelihood set for a haplotype group is generated given the observed data (e.g., allele quantifications). In some embodiments, a haplotype pair likelihood set for a haplotype group is generated, given the observed data (e.g., allele quantifications), according to i) allele quantifications and ii) a probability of each haplotype pair. In some embodiments, a haplotype pair likelihood set for a haplotype group is generated, given the observed data (e.g., allele quantifications), according to i) a probability of the observed data (e.g., allele quantifications) given each haplotype pair, and ii) a probability of each haplotype pair. In some embodiments, a haplotype pair likelihood set for a haplotype group is generated, given the allele quantifications, according to i) a probability of the allele quantifications given each haplotype pair, and ii) a probability of each haplotype pair. In some embodiments, the probability in (i) is determined according to which genotype is most likely observed at each genomic locus across a haplotype group, given a particular haplotype pair. In some embodiments, a method herein comprises calculating the probability of the allele quantifications at each at each genomic locus and generating a product across all genomic loci in the haplotype group. In some embodiments, the probability in (i) is adjusted according to a measure of sequencing error, as described herein.

In some embodiments, a probability of each haplotype pair is determined, in part, according to haplotype frequencies (e.g., haplotype frequencies described herein, haplotype frequencies in a population, haplotype frequencies in a database). In some embodiments, a probability of each haplotype pair is determined, in part, according to haplotype frequencies for each haplotype species (e.g., for haplotype pair AB, the frequency of haplotype species A in a population and the frequency of haplotype species B in a population). In some embodiments, a probability of each haplotype pair is determined, in part, according to haplotype frequencies for (i) a target reference allele and a linked reference allele, (ii) a target reference allele and a linked alternative allele, (iii) a target alternative allele and a linked reference allele, and (iv) a target alternative allele and a linked alternative allele. In some embodiments, a probability of each haplotype pair is determined, in part, according to haplotype frequencies for (i) a first linked reference allele and a second linked reference allele, (ii) a first linked reference allele and a second linked alternative allele, (iii) a first linked alternative allele and a second linked reference allele, and (iv) a first linked alternative allele and a second linked alternative allele.

In some embodiments, a haplotype pair likelihood set for a haplotype group is generated according to a probability that the test sample has a particular haplotype pair, i and j, given the allele quantifications in (b), D, where the probability, P(Hᵢ, Hⱼ|D), is derived from equation A:

$$P ( {H}_{i} , {H}_{j} \vert D ) = \frac{P ( D \vert {H}_{i} , {H}_{j} ) \times P ( {H}_{i} , {H}_{j} )}{P ( D )} \qquad ( A )$$

where P(D|Hᵢ, Hⱼ) is the probability of the allele quantifications, given the allele quantifications derive from haplotype pair Hᵢ, Hⱼ; P(Hᵢ, Hⱼ) is the probability of each haplotype pair derived from haplotype frequencies; and P(D) is the probability of the data (allele quantifications). Generally it is not necessary to explicitly calculate P(D) because this term is cancelled out when the ratios of P(Hᵢ, Hⱼ|D) are taken later. In some embodiments, P(D|Hᵢ, Hⱼ) is determined according to which genotype is most likely observed at each genomic locus, s, across the haplotype group, given haplotype pair Hᵢ, Hⱼ is present.

In some embodiments, a method herein comprises calculating the probability of the allele quantifications, D, at each at each genomic locus, s, and generating a product across all genomic loci in the haplotype group according to equation B:

$$P ( D \vert {H}_{i} , {H}_{j} ) = {\prod }_{s = 1}^{n} P ( {D}_{s} \vert {H}_{i s} , {H}_{j s} ) . \qquad ( B )$$

A method herein may comprise generating a genotype at a target genomic locus. A method herein may comprise generating a genotype at a target genomic locus based on a haplotype pair likelihood set. A genotype at a target genomic locus may be chosen from homozygous for a target reference allele, heterozygous for a target reference allele and a target alternative allele, and homozygous for a target alternative allele. In some embodiments, a method herein comprises identifying the most probable haplotype pair from the haplotype pair likelihood set. A genotype at a target genomic locus may be generated according to the most probable haplotype pair. For example, a most probable haplotype pair may comprise a specific allele at a target locus in a first haplotype species in the pair and specific allele at a target locus in a second haplotype species in the pair. Thus, the genotype at a target genomic locus is both target alleles ([reference, reference]; [reference, alternative]; or [alternative, alternative]) in the selected haplotype pair. In some embodiments, a method herein comprises aggregating haplotype pair likelihoods across all haplotype pairs for the haplotype group, thereby generating aggregate likelihoods. For example, each haplotype pair corresponds to one of three possible target genomic locus genotypes (homozygous reference, heterozygous, and homozygous alternative). The probability of the data (e.g., allele quantifications) given a haplotype pair, calculated as described above, may be added to an aggregate probability of the corresponding target genomic locus genotype. A genotype at a target genomic locus may be generated according to the highest aggregate likelihood.

In some embodiments, a plurality of genotypes at a plurality of target genomic loci are generated. In some embodiments, a plurality of genotypes at a plurality of target genomic loci are generated based on a plurality of haplotype pair likelihood sets. In some embodiments, a method herein comprises identifying a subject based on a plurality of genotypes generated for a test sample, as described herein.

Samples

Provided herein are methods and compositions for processing and/or analyzing nucleic acid. Nucleic acid or a nucleic acid mixture utilized in methods and compositions described herein may be isolated from a sample obtained from a subject (e.g., a test subject). A subject can be any living or non-living organism, including but not limited to a human, a non-human animal, a plant, a bacterium, a fungus, a protist or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. A subject may be a patient having an infection or infectious disease or infected with a pathogen (e.g., bacteria, virus, fungus, protozoa, and the like), a patient suspected of having an infection or infectious disease or being infected with a pathogen, a patient recovering from an infection, infectious disease, or pathogenic infection, a patient with a history of infections, infectious disease, pathogenic infections, and/or a subject obtaining an infectious disease or pathogen screen. A subject may be a transplant recipient. A subject may be a patient undergoing a microbiome analysis. In some embodiments, a test subject is a female. In some embodiments, a test subject is a human female. In some embodiments, a test subject is a male. In some embodiments, a test subject is a human male.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism (e.g., bacteria, yeast, or the like). In some embodiments, a nucleic acid sample is isolated or obtained from a cell(s), tissue, organ, and/or the like of an animal (e.g., an animal subject). In some embodiments, a nucleic acid sample is isolated or obtained from a source such as bacteria, yeast, insects (e.g., drosophila), mammals, amphibians (e.g., frogs (e.g., Xenopus)), viruses, plants, or any other mammalian or non-mammalian nucleic acid sample source.

A nucleic acid sample may be isolated or obtained from an extant organism or animal. In some instances, a nucleic acid sample may be isolated or obtained from an extinct (or “ancient”) organism or animal (e.g., an extinct mammal; an extinct mammal from the genus Homo). In some instances, a nucleic acid sample may be obtained as part of a diagnostic analysis.

In some instances, a nucleic acid sample may be obtained as part of a forensics analysis. In some embodiments, a genotyping method and/or a genealogy analysis described herein is applied to a forensic sample or specimen (e.g., a sample or specimen associated with a crime; unidentified remains associated with a crime). A forensic sample or specimen may include any biological substance that contains nucleic acid. For example, a forensic sample or specimen may include blood, semen, hair, skin, sweat, saliva, decomposed tissue, bone, fingernail scrapings, licked stamps/envelopes, sluff, touch DNA, razor residue, and the like. In some embodiments, a forensic sample or specimen comprises hair or hair fragments. In some embodiments, a forensic sample or specimen comprises bone or bone fragments.

In some embodiments, a genotyping method and/or a genealogy analysis described herein is applied to a non-forensic sample or specimen (e.g., a sample or specimen that is not associated with a crime; unidentified remains not associated with a crime; historical objects containing biological material of deceased (e.g., for genealogy purposes)). A non-forensic sample or specimen may include any biological substance that contains nucleic acid. For example, a non-forensic sample or specimen may include blood, semen, hair, skin, sweat, saliva, decomposed tissue, bone, fingernail scrapings, licked stamps/envelopes, sluff, touch DNA, razor residue, and the like. In some embodiments, a non-forensic sample or specimen comprises hair or hair fragments. In some embodiments, a non-forensic sample or specimen comprises bone or bone fragments.

A sample or test sample may be any specimen that is isolated or obtained from a subject or part thereof (e.g., a human subject, a pregnant female, a cancer patient, a patient having an infection or infectious disease, a transplant recipient, a fetus, a tumor, an infected organ or tissue, a transplanted organ or tissue, a microbiome). A sample sometimes is from a pregnant female subject bearing a fetus at any stage of gestation (e.g., first, second or third trimester for a human subject), and sometimes is from a post-natal subject. A sample sometimes is from a pregnant subject bearing a fetus that is euploid for all chromosomes, and sometimes is from a pregnant subject bearing a fetus having a chromosome aneuploidy (e.g., one, three (i.e., trisomy (e.g., T21, T18, T13)), or four copies of a chromosome) or other genetic variation. Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, fetal cells or cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebral spinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of fetal DNA found in maternal blood, for example, may be performed using, e.g., whole blood, serum, or plasma. An analysis of tumor or cancer DNA found in a patient's blood, for example, may be performed using, e.g., whole blood, serum, or plasma. An analysis of pathogen DNA found in a patient's blood, for example, may be performed using, e.g., whole blood, serum, or plasma. An analysis of transplant DNA found in a transplant recipient's blood, for example, may be performed using, e.g., whole blood, serum, or plasma. Methods for preparing serum or plasma from blood obtained from a subject (e.g., a maternal subject; patient; cancer patient) are known. For example, a subject's blood (e.g., a pregnant woman's blood; patient's blood; cancer patient's blood) can be placed in a tube containing EDTA or a specialized commercial product such as Cell-Free DNA BCT (Streck, Omaha, Nebr.) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation. Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

A sample may be heterogeneous. For example, a sample may include more than one cell type and/or one or more nucleic acid species. In some instances, a sample may include (i) fetal cells and maternal cells, (ii) cancer cells and non-cancer cells, and/or (iii) pathogenic cells and host cells. In some instances, a sample may include (i) cancer and non-cancer nucleic acid, (ii) pathogen and host nucleic acid, (iii) fetal derived and maternal derived nucleic acid, and/or more generally, (iv) mutated and wild-type nucleic acid. In some instances, a sample may include a minority nucleic acid species and a majority nucleic acid species, as described in further detail below. In some instances, a sample may include cells and/or nucleic acid from a single subject or may include cells and/or nucleic acid from multiple subjects.

In some embodiments, a sample comprises double-stranded nucleic acid fragments. In some embodiments, a sample comprises single-stranded nucleic acid fragments. In some embodiments, a sample comprises double-stranded nucleic acid fragments and single-stranded nucleic acid fragments.

Nucleic Acid

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single-stranded form or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame), and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single-stranded or double-stranded, or may be a mixture of single-stranded and double-stranded. Single stranded DNA (ssDNA), for example, can be generated by denaturing double stranded DNA by heating or by treatment with alkali, for example. Accordingly, in some embodiments, ssDNA is derived from double-stranded DNA (dsDNA). In some embodiments, a method herein comprises prior to combining a nucleic acid composition comprising dsDNA with sequencing adapters, denaturing the dsDNA, thereby generating ssDNA.

In certain embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

Nucleic acid (e.g., nucleic acid targets, single-stranded nucleic acid (ssNA), polynucleotides, oligonucleotides, overhangs, hybridization regions) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). In some embodiments, one or more thymine (T) bases are replaced by uracil (U) in a sequencing adapter, and is/are complementary to adenine (A). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions.

In certain instances, a nucleotide sequence may be partially complementary to a target, in which not all nucleotides are complementary to every nucleotide in the target nucleic acid in all the corresponding positions. For example, a hybridization region may be perfectly (i.e., 100%) complementary to a target region, or a hybridization region may share some degree of complementarity which is less than perfect (e.g., 70%, 75%, 85%, 90%, 95%, 99%). In another example, a hybridization region may be perfectly (i.e., 100%) complementary to an oligonucleotide, or a hybridization region may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position.

In some embodiments, nucleic acids in a mixture of nucleic acids are analyzed. A mixture of nucleic acids can comprise two or more nucleic acid species having the same or different nucleotide sequences, different lengths, different origins (e.g., genomic origins, fetal vs. maternal origins, cell or tissue origins, cancer vs. non-cancer origin, tumor vs. non-tumor origin, host vs. pathogen, host vs. transplant, host vs. microbiome, sample origins, subject origins, and the like), different overhang lengths, different overhang types (e.g., 5′ overhangs, 3′ overhangs, no overhangs), or combinations thereof. In some embodiments, a mixture of nucleic acids comprises single-stranded nucleic acid and double-stranded nucleic acid. In some embodiment, a mixture of nucleic acids comprises DNA and RNA. In some embodiment, a mixture of nucleic acids comprises ribosomal RNA (rRNA) and messenger RNA (mRNA). Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

In some embodiments, target nucleic acids comprise damaged or degraded nucleic acid. Damaged or degraded nucleic acid may be referred to as low-quality nucleic acid or highly damaged/degraded nucleic acid. Damaged or degraded nucleic acid may be highly fragmented, and may include damage such as base analogs and abasic sites subject to miscoding lesions and/or intermolecular crosslinking. For example, sequencing errors resulting from deamination of cytosine residues may be present in certain sequences obtained from damaged or degraded DNA (e.g., miscoding of C to T and G to A). In some embodiments, target nucleic acids are derived from nicked double-stranded nucleic acid fragments. Nicked double-stranded nucleic acid fragments may be denatured (e.g., heat denatured) to generate ssNA fragments.

Nucleic acid may be derived from one or more sources (e.g., biological sample, blood, cells, serum, plasma, buffy coat, urine, lymphatic fluid, skin, hair, bone, soil, and the like) by methods known in the art. In some embodiments, nucleic acid may be derived from a forensic sample or specimen. In some embodiments, a genotyping method and/or a genealogy analysis described herein is applied to nucleic acid derived from a forensic sample or specimen. In some embodiments, nucleic acid derived from a forensic sample or specimen comprises damaged, degraded, and/or fragmented nucleic acid. In some embodiments, nucleic acid is derived from a forensic sample or specimen comprises comprising no living cells. Nucleic acid derived from a forensic sample or specimen may include nucleic acid derived from blood, semen, hair, skin, sweat, saliva, decomposed tissue, bone, fingernail scrapings, licked stamps/envelopes, sluff, touch DNA, razor residue, and the like. In some embodiments, nucleic acid derived from a forensic sample or specimen comprises nucleic acid derived from hair or hair fragments. In some embodiments, nucleic acid derived from a forensic sample or specimen comprises nucleic acid derived from hair or hair fragments, where the hair or hair fragments comprise no roots or living cells. In some embodiments, nucleic acid derived from a forensic sample or specimen comprises nucleic acid derived from bone or bone fragments. In some embodiments, nucleic acid derived from a forensic sample or specimen comprises nucleic acid derived from bone or bone fragments, where the bone or bone fragments comprise no living cells.

Any suitable method can be used for isolating, extracting and/or purifying DNA from a biological sample (e.g., from blood or a blood product), non-limiting examples of which include methods of DNA preparation (e.g., described by Sambrook and Russell, Molecular Cloning: A Laboratory Manual 3d ed., 2001), various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md.); GenomicPrep™ Blood DNA Isolation Kit (Promega, Madison, Wis.); GFX™ Genomic Blood DNA Purification Kit (Amersham, Piscataway, N.J.); DNAzol®, ChargeSwitch®, Purelink®, GeneCatcher® nucleic acid isolation/purification kits by Life Technologies, Inc. (Carlsbad, Calif.); NucleoMag®, NucleoSpin®, and NucleoBond® nucleic acid isolation/purification kits by Clontech Laboratories, Inc. (Mountain View, Calif.); the like or combinations thereof. In certain aspects, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md.), the RecoverAll® Total Nucleic Acid Isolation kit for FFPE by Life Technologies, Inc. (Carlsbad, Calif.), and the NucleoSpin® FFPE kits by Clontech Laboratories, Inc. (Mountain View, Calif.).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCI), sodium acetate) and isopropanol, and binding DNA in this solution to silica-based column. In some instances, a lysis protocol includes certain procedures described in Dabney et al., Proceedings of the National Academy of Sciences 110, no. 39 (2013): 15758-15763.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccf DNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In some embodiments, cell-free nucleic acid comprises cell-free fetal nucleic acid (e.g., cell-free fetal DNA). In certain aspects, cell-free nucleic acid comprises circulating cancer nucleic acid (e.g., cancer DNA). In certain aspects, cell-free nucleic acid comprises circulating tumor nucleic acid (e.g., tumor DNA). In some embodiments, cell-free nucleic acid comprises infectious agent nucleic acid (e.g., pathogen DNA). In some embodiments, cell-free nucleic acid comprises nucleic acid (e.g., DNA) from a transplant. In some embodiments, cell-free nucleic acid comprises nucleic acid (e.g., DNA) from a microbiome (e.g., microbiome of gut, microbiome of blood, microbiome of mouth, microbiome of spinal fluid, microbiome of feces).

Cell-free DNA (cfDNA) may originate from degraded sources and often provides limiting amounts of DNA when extracted. Certain methods for generating nucleic acid libraries (e.g., methods for generating sequencing libraries described in International Patent Application Publication No. WO 2019/140201, U.S. Provisional Patent Application No. 62/830,211, and U.S. Provisional Patent Application No. 62/861,594, each of which is incorporated by reference herein) are able to capture a larger amount of short DNA fragments from cfDNA. cfDNA from cancer samples, for example, tends to have a higher population of short fragments. In certain instances, short fragments in cfDNA may be enriched for fragments originating from transcription factors rather than nucleosomes.

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In another example, blood serum or plasma from a pregnant female can include maternal nucleic acid and fetal nucleic acid. In another example, blood serum or plasma from a patient having an infection or infectious disease can include host nucleic acid and infectious agent or pathogen nucleic acid. In another example, a sample from a subject having received a transplant can include host nucleic acid and nucleic acid from the donor organ or tissue. In some instances, cancer nucleic acid, tumor nucleic acid, fetal nucleic acid, pathogen nucleic acid, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, tumor, fetal, pathogen, transplant, or microbiome nucleic acid). In another example, heterogeneous nucleic acid may include nucleic acid from two or more subjects (e.g., a sample from a crime scene).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. For example, fetal nucleic acid can be purified from a mixture comprising maternal and fetal nucleic acid. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, small fragments of fetal nucleic acid (e.g., 30 to 500 bp fragments) can be purified, or partially purified, from a mixture comprising both fetal and maternal nucleic acid fragments. In certain examples, nucleosomes comprising smaller fragments of fetal nucleic acid can be purified from a mixture of larger nucleosome complexes comprising larger fragments of maternal nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

Nucleic acids may be amplified under amplification conditions. The term “amplified” or “amplification” or “amplification conditions” as used herein refers to subjecting a target nucleic acid in a sample or a nucleic acid product generated by a method herein to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). In certain instances, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule).

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any suitable form useful for conducting a sequence analysis.

In some embodiments, target nucleic acids (e.g., ssNAs, dsNAs, or a combination thereof) are not modified prior to combining with sequencing adapters. In some embodiments, target nucleic acids are not modified in length prior to combining with sequencing adapters. In this context, “not modified” means that target nucleic acids are isolated from a sample and then combined with sequencing adapters, without modifying the length or the composition of the target nucleic acids. For example, target nucleic acids may not be shortened (e.g., they are not contacted with a restriction enzyme or nuclease or physical condition that reduces length (e.g., shearing condition, cleavage condition)) and may not be increased in length by one or more nucleotides (e.g., ends are not filled in at overhangs; no nucleotides are added to the ends). Adding a phosphate or chemically reactive group to one or both ends of a target nucleic acid generally is not considered modifying the nucleic acid or modifying the length of the nucleic acid. Denaturing a double-stranded nucleic acid (dsNA) fragment to generate an ssNA fragment generally is not considered modifying the nucleic acid or modifying the length of the nucleic acid.

In some embodiments, one or both native ends of target nucleic acids (e.g., ssNAs, dsNAs, or a combination thereof) are present when the target nucleic acid is combined with sequencing adapters. Native ends generally refer to unmodified ends of a nucleic acid fragment. In some embodiments, native ends of target nucleic acids are not modified in length prior to combining with sequencing adapters. In this context, “not modified” means that target nucleic acids are isolated from a sample and then combined with sequencing adapters, or components thereof, without modifying the length of the native ends of target nucleic acids. For example, target nucleic acids are not shortened (e.g., they are not contacted with a restriction enzyme or nuclease or physical condition that reduces length (e.g., shearing condition, cleavage condition) to generate non-native ends) and are not increased in length by one or more nucleotides (e.g., native ends are not filled in at overhangs; no nucleotides are added to the native ends). Adding a phosphate or chemically reactive group to one or both native ends of a target nucleic acid generally is not considered modifying the length of the nucleic acid.

In some embodiments, target nucleic acids (e.g., ssNAs, dsNAs, or a combination thereof) are not contacting with a cleavage agent (e.g., endonuclease, exonuclease, restriction enzyme) and/or a polymerase prior to combining with sequencing adapters. In some embodiments, target nucleic acids are not subjected to mechanical shearing (e.g., ultrasonication (e.g., Adaptive Focused Acoustics™ (AFA) process by Covaris)) prior to combining with sequencing adapters. In some embodiments, target nucleic acids are not contacting with an exonuclease (e.g., DNAse) prior to combining with sequencing adapters. In some embodiments, target nucleic acids are not amplified prior to combining with sequencing adapters. In some embodiments, target nucleic acids are not attached to a solid support prior to combining with sequencing adapters. In some embodiments, target nucleic acids are not conjugated to another molecule prior to combining with sequencing adapters. In some embodiments, target nucleic acids are not cloned into a vector prior to combining with sequencing adapters. In some embodiments, target nucleic acids may be subjected to dephosphorylation prior to combining with sequencing adapters. In some embodiments, target nucleic acids may be subjected to phosphorylation prior to combining with sequencing adapters.

In some embodiments, combining target nucleic acids (e.g., ssNAs, dsNAs, or a combination thereof) with sequencing adapters, comprises isolating the target nucleic acids, and combining the isolated target nucleic acids with sequencing adapters. In some embodiments, combining target nucleic acids with sequencing adapters comprises isolating the target nucleic acids, phosphorylating the isolated target nucleic acids, and combining the phosphorylated target nucleic acids with sequencing adapters. In some embodiments, combining target nucleic acids with sequencing adapters comprises isolating the target nucleic acids, dephosphorylating the sequencing adapters and combining the isolated target nucleic acids with the dephosphorylated sequencing adapters. In some embodiments, combining target nucleic acids with sequencing adapters comprises isolating the target nucleic acids, dephosphorylating the isolated target nucleic acids, phosphorylating the dephosphorylated target nucleic acids, and combining the phosphorylated target nucleic acids with sequencing adapters. In some embodiments, combining target nucleic acids with sequencing adapters comprises isolating the target nucleic acids, dephosphorylating the isolated target nucleic acids, phosphorylating the dephosphorylated target nucleic acids, dephosphorylating the sequencing adapters, and combining the phosphorylated target nucleic acids with the dephosphorylated sequencing adapters.

In some embodiments, combining target nucleic acids (e.g., ssNAs, dsNAs, or a combination thereof) with sequencing adapters consists of isolating the target nucleic acids, and combining the isolated target nucleic acids with sequencing adapters. In some embodiments, combining target nucleic acids with sequencing adapters consists of isolating the target nucleic acids, phosphorylating the isolated target nucleic acids, and combining the phosphorylated target nucleic acids with sequencing adapters. In some embodiments, combining target nucleic acids with sequencing adapters consists of isolating the target nucleic acids, dephosphorylating the sequencing adapters, and combining the isolated target nucleic acids with the dephosphorylated sequencing adapters. In some embodiments, combining target nucleic acids with sequencing adapters consists of isolating the target nucleic acids, dephosphorylating the isolated target nucleic acids, phosphorylating the dephosphorylated target nucleic acids, and combining the phosphorylated target nucleic acids with sequencing adapters. In some embodiments, combining target nucleic acids with sequencing adapters consists of isolating the target nucleic acids, dephosphorylating the isolated target nucleic acids, phosphorylating the dephosphorylated target nucleic acids, dephosphorylating the sequencing adapters, and combining the phosphorylated target nucleic acids with the dephosphorylated sequencing adapters.

Enriching Nucleic Acids

In some embodiments, nucleic acid (e.g., extracellular nucleic acid; sample nucleic acid; target nucleic acid (e.g., ssNA, dsNA, or a combination thereof)) is enriched or relatively enriched for a subpopulation or species of nucleic acid. Nucleic acid subpopulations can include, for example, fetal nucleic acid, maternal nucleic acid, cancer nucleic acid, tumor nucleic acid, patient nucleic acid, host nucleic acid, pathogen nucleic acid, transplant nucleic acid, microbiome nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with a method provided herein. Thus, in certain embodiments, methods of the technology comprise an additional step of enriching for a subpopulation of nucleic acid in a sample. In certain embodiments, nucleic acid from normal tissue (e.g., non-cancer cells, host cells) is selectively removed (partially, substantially, almost completely or completely) from the sample. In certain embodiments, maternal nucleic acid is selectively removed (partially, substantially, almost completely or completely) from the sample. In certain embodiments, enriching for a particular low copy number species nucleic acid (e.g., cancer, tumor, fetal, pathogen, transplant, microbiome nucleic acid) may improve quantitative sensitivity. Methods for enriching a sample for a particular species of nucleic acid are described, for example, in U.S. Pat. No. 6,927,028, International Patent Application Publication No. WO2007/140417, International Patent Application Publication No. WO2007/147063, International Patent Application Publication No. WO2009/032779, International Patent Application Publication No. WO2009/032781, International Patent Application Publication No. WO2010/033639, International Patent Application Publication No. WO2011/034631, International Patent Application Publication No. WO2006/056480, and International Patent Application Publication No. WO2011/143659, the entire content of each is incorporated herein by reference, including all text, tables, equations and drawings.

In some embodiments, nucleic acid is enriched for certain target fragment species and/or reference fragment species. In certain embodiments, nucleic acid is enriched for a specific nucleic acid fragment length or range of fragment lengths using one or more length-based separation methods described below. In certain embodiments, nucleic acid is enriched for fragments from a select genomic region (e.g., chromosome) using one or more sequence-based separation methods described herein and/or known in the art.

Non-limiting examples of methods for enriching for a nucleic acid subpopulation in a sample include methods that exploit epigenetic differences between nucleic acid species (e.g., methylation-based fetal nucleic acid enrichment methods described in U.S. Patent Application Publication No. 2010/0105049, which is incorporated by reference herein); restriction endonuclease enhanced polymorphic sequence approaches (e.g., such as a method described in U.S. Patent Application Publication No. 2009/0317818, which is incorporated by reference herein); selective enzymatic degradation approaches; massively parallel signature sequencing (MPSS) approaches; amplification (e.g., PCR)-based approaches (e.g., loci-specific amplification methods, multiplex SNP allele PCR approaches; universal amplification methods); pull-down approaches (e.g., biotinylated ultramer pull-down methods); extension and ligation-based methods (e.g., molecular inversion probe (MIP) extension and ligation); and combinations thereof.

In some embodiments, nucleic acid is enriched for fragments from a select genomic region (e.g., chromosome) using one or more sequence-based separation methods described herein. Sequence-based separation generally is based on nucleotide sequences present in the fragments of interest (e.g., target and/or reference fragments) and substantially not present in other fragments of the sample or present in an insubstantial amount of the other fragments (e.g., 5% or less). In some embodiments, sequence-based separation can generate separated target fragments and/or separated reference fragments. Separated target fragments and/or separated reference fragments often are isolated away from the remaining fragments in the nucleic acid sample. In certain embodiments, the separated target fragments and the separated reference fragments also are isolated away from each other (e.g., isolated in separate assay compartments). In certain embodiments, the separated target fragments and the separated reference fragments are isolated together (e.g., isolated in the same assay compartment). In some embodiments, unbound fragments can be differentially removed or degraded or digested.

In some embodiments, a selective nucleic acid capture process is used to separate target and/or reference fragments away from a nucleic acid sample. Commercially available nucleic acid capture systems include, for example, NIMBLEGEN sequence capture system (Roche NIMBLEGEN, Madison, Wis.); ILLUMINA BEADARRAY platform (Illumina, San Diego, Calif.); Affymetrix GENECHIP platform (Affymetrix, Santa Clara, Calif.); Agilent SURESELECT Target Enrichment System (Agilent Technologies, Santa Clara, Calif.); and related platforms. Such methods typically involve hybridization of a capture oligonucleotide to a part or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci, or a particular sequence in a nucleic acid target. In certain embodiments, a hybridization-based method (e.g., using oligonucleotide arrays) can be used to enrich for fragments containing certain nucleic acid sequences. Thus, in some embodiments, a nucleic acid sample is optionally enriched by capturing a subset of fragments using capture oligonucleotides complementary to, for example, selected sequences in sample nucleic acid. In certain instances, captured fragments are amplified. For example, captured fragments containing adapters may be amplified using primers complementary to the adapter sequences to form collections of amplified fragments, indexed according to adapter sequence. In some embodiments, nucleic acid is enriched for fragments from a select genomic region (e.g., chromosome, a gene) by amplification of one or more regions of interest using oligonucleotides (e.g., PCR primers) complementary to sequences in fragments containing the region(s) of interest, or part(s) thereof.

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In certain instances, length-based separation approaches can include selective sequence tagging approaches, fragment circularization, chemical treatment (e.g., formaldehyde, polyethylene glycol (PEG) precipitation), mass spectrometry and/or size-specific nucleic acid amplification, for example.

In some embodiments, a method herein includes enriching an RNA species in a mixture of RNA species. For example, a method herein may comprise enriching messenger RNA (mRNA) present in a mixture of mRNA and ribosomal RNA (rRNA). Any suitable mRNA enrichment method may be used, which includes rRNA depletion and/or mRNA enrichment methods such as rRNA depletion with magnetic beads (e.g., Ribo-Zero™, Ribominus™, and MICROBExpress™, which use rRNA depletion probes in combination with magnetic beads to deplete rRNAs from a sample, thus enriching mRNAs), oligo(dT)-based poly(A) enrichment (e.g., BioMag® Oligo (dT)20), nuclease-based rRNA depletion (e.g., digestion of rRNA with Terminator™ 5′-Phosphate Dependent Exonuclease), and combinations thereof.

Length-Based Separation

In some embodiments, a method herein comprises separating target nucleic acids (e.g., ssNAs, dsNAs, or a combination thereof) according to fragment length. For example, target nucleic acids may be enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also may be referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In some embodiments, length-based separation approaches can include fragment circularization, chemical treatment (e.g., formaldehyde, polyethylene glycol (PEG)), mass spectrometry and/or size-specific nucleic acid amplification, for example. In some embodiments, length based-separation is performed using Solid Phase Reversible Immobilization (SPRI) beads.

In some embodiments, nucleic acid fragments of a certain length, range of lengths, or lengths under or over a particular threshold or cutoff are separated from the sample. In some embodiments, fragments having a length under a particular threshold or cutoff (e.g., 500 bp, 400 bp, 300 bp, 200 bp, 150 bp, 100 bp) are referred to as “short” fragments and fragments having a length over a particular threshold or cutoff (e.g., 500 bp, 600 bp, 700 bp, 800 bp, 900 bp, 1000 bp) are referred to as “long” fragments, large fragments, and/or high molecular weight (HMW) fragments. In some embodiments, fragments of a certain length, range of lengths, or lengths under or over a particular threshold or cutoff are retained for analysis while fragments of a different length or range of lengths, or lengths over or under the threshold or cutoff are not retained for analysis. In some embodiments, fragments that are less than about 500 bp are retained. In some embodiments, fragments that are less than about 400 bp are retained. In some embodiments, fragments that are less than about 300 bp are retained. In some embodiments, fragments that are less than about 200 bp are retained. In some embodiments, fragments that are less than about 150 bp are retained. For example, fragments that are less than about 190 bp, 180 bp, 170 bp, 160 bp, 150 bp, 140 bp, 130 bp, 120 bp, 110 bp or 100 bp are retained. In some embodiments, fragments that are about 100 bp to about 200 bp are retained. For example, fragments that are about 190 bp, 180 bp, 170 bp, 160 bp, 150 bp, 140 bp, 130 bp, 120 bp or 110 bp are retained. In some embodiments, fragments that are in the range of about 100 bp to about 200 bp are retained. For example, fragments that are in the range of about 110 bp to about 190 bp, 130 bp to about 180 bp, 140 bp to about 170 bp, 140 bp to about 150 bp, 150 bp to about 160 bp, or 145 bp to about 155 bp are retained.

In some embodiments, target nucleic acids (e.g., ssNAs, dsNAs, or a combination thereof) having fragment lengths of less than about 1000 bp are combined with a plurality or pool of sequencing adapters. In some embodiments, target nucleic acids having fragment lengths of less than about 500 bp are combined with a plurality or pool of sequencing adapters. In some embodiments, target nucleic acids having fragment lengths of less than about 400 bp are combined with a plurality or pool of sequencing adapters. In some embodiments, target nucleic acids having fragment lengths of less than about 300 bp are combined with a plurality or pool of sequencing adapters. In some embodiments, target nucleic acids having fragment lengths of less than about 200 bp are combined with a plurality or pool of sequencing adapters. In some embodiments, target nucleic acids having fragment lengths of less than about 100 bp are combined with a plurality or pool of sequencing adapters.

In some embodiments, target nucleic acids (e.g., ssNAs, dsNAs, or a combination thereof) having fragment lengths of about 100 bp or more are combined with a plurality or pool of sequencing adapters. In some embodiments, target nucleic acids having fragment lengths of about 200 bp or more are combined with a plurality or pool of sequencing adapters. In some embodiments, target nucleic acids having fragment lengths of about 300 bp or more are combined with a plurality or pool of sequencing adapters. In some embodiments, target nucleic acids having fragment lengths of about 400 bp or more are combined with a plurality or pool of sequencing adapters. In some embodiments, target nucleic acids having fragment lengths of about 500 bp or more are combined with a plurality or pool of sequencing adapters. In some embodiments, target nucleic acids having fragment lengths of about 1000 bp or more are combined with a plurality or pool of sequencing adapters.

In some embodiments, target nucleic acids (e.g., ssNAs, dsNAs, or a combination thereof) having any fragment length or any combination of fragment lengths are combined with a plurality or pool of sequencing adapters. For example, target nucleic acids having fragment lengths of less than 500 bp and fragments lengths of 500 bp or more may be combined with a plurality or pool of sequencing adapters.

Certain length-based separation methods that can be used with methods described herein employ a selective sequence tagging approach, for example. In such methods, a fragment size species (e.g., short fragments) nucleic acids are selectively tagged in a sample that includes long and short nucleic acids. Such methods typically involve performing a nucleic acid amplification reaction using a set of nested primers which include inner primers and outer primers. In some embodiments, one or both of the inner can be tagged to thereby introduce a tag onto the target amplification product. The outer primers generally do not anneal to the short fragments that carry the (inner) target sequence. The inner primers can anneal to the short fragments and generate an amplification product that carries a tag and the target sequence. Typically, tagging of the long fragments is inhibited through a combination of mechanisms which include, for example, blocked extension of the inner primers by the prior annealing and extension of the outer primers. Enrichment for tagged fragments can be accomplished by any of a variety of methods, including for example, exonuclease digestion of single-stranded nucleic acid and amplification of the tagged fragments using amplification primers specific for at least one tag.

Another length-based separation method that can be used with methods described herein involves subjecting a nucleic acid sample to polyethylene glycol (PEG) precipitation. Examples of methods include those described in International Patent Application Publication Nos. WO2007/140417 and WO2010/115016. This method in general entails contacting a nucleic acid sample with PEG in the presence of one or more monovalent salts under conditions sufficient to substantially precipitate large nucleic acids without substantially precipitating small (e.g., less than 300 nucleotides) nucleic acids.

Another length-based enrichment method that can be used with methods described herein involves circularization by ligation, for example, using circligase. Short nucleic acid fragments typically can be circularized with higher efficiency than long fragments. Non-circularized sequences can be separated from circularized sequences, and the enriched short fragments can be used for further analysis.

Nucleic Acid Library

Methods herein may include preparing a nucleic acid library and/or modifying nucleic acids for a nucleic acid library. In some embodiments, ends of nucleic acid fragments are modified such that the fragments, or amplified products thereof, may be incorporated into a nucleic acid library. Generally, a nucleic acid library refers to a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In some embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component Clq, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI) described herein, a palindromic sequence described herein, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adapters (e.g., oligonucleotide adapters described herein). In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments hybridized oligonucleotides are labeled probes. In some embodiments, a library of nucleic acids comprises hybridized oligonucleotide probes prior to immobilization on a solid phase.

In some embodiments, a polynucleotide of known sequence comprises a universal sequence. A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation (e.g., when using cell-free DNA).

In certain embodiments, a ligation-based library preparation method is used (e.g., ILLUMINA TRUSEQ, Illumina, San Diego Calif.). Ligation-based library preparation methods often make use of an adapter (e.g., a methylated adapter) design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids (e.g., fragmented nucleic acids or cell-free DNA) may be end repaired by a fill-in reaction, an exonuclease reaction or a combination thereof. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides. In some embodiments, end repair is omitted and sequencing adapters are ligated directly to the native ends of nucleic acids (e.g., double-stranded nucleic acids, single-stranded nucleic acids, fragmented nucleic acids, and/or cell-free nucleic acids).

In some embodiments, nucleic acid library preparation comprises ligating a sequencing adapter, or component thereof, to a nucleic acid (e.g., to a sample nucleic acid, to a sample nucleic acid fragment, to a template nucleic acid, to a target nucleic acid). Examples of adapters useful for generating a nucleic acid library (e.g., a sequencing library) are described in International Patent Application Publication No. WO 2018/013837, International Patent Application Publication No. WO 2019/140201, International Patent Application Publication No. WO2019/236726, and International Patent Application Publication No. WO/2020/206143, each of which is incorporated by reference herein.

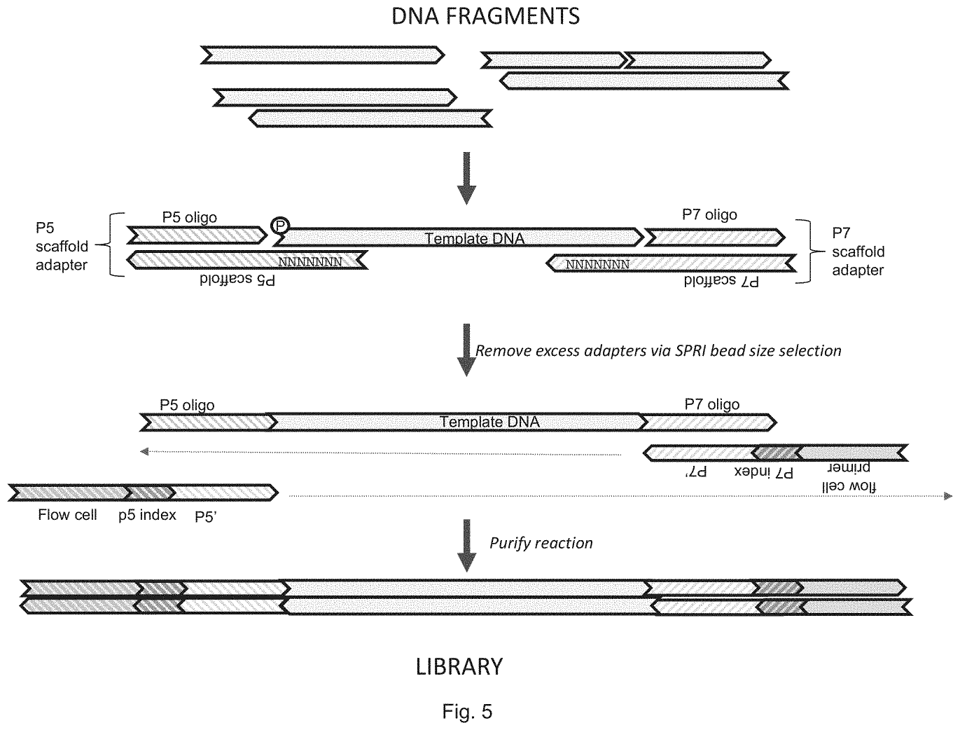

In some embodiments, a nucleic acid library preparation comprises ligating a scaffold adapter, or component thereof, to a nucleic acid (e.g., to a sample nucleic acid, to a sample nucleic acid fragment, to a template nucleic acid, to a target nucleic acid). In some embodiments, a nucleic acid library preparation comprises ligating a scaffold adapter, or component thereof, to a single-stranded nucleic acid (ssNA). Scaffold adapters generally include a scaffold polynucleotide and an oligonucleotide. Accordingly, a “component” of a scaffold adapter may refer to a scaffold polynucleotide and/or an oligonucleotide, or a subcomponent or region thereof. An example of a scaffold adapter is provided in FIG. 5. The oligonucleotide and/or the scaffold polynucleotide can be composed of pyrimidine (C, T, U) and/or purine (A, G) nucleotides. Additional components or subcomponents may include one or more of an index polynucleotide, a unique molecular identifier (UMI), primer binding site (e.g., P5 primer binding site, P7 primer binding site), flow cell binding region, and the like, and complements thereto.

A scaffold polynucleotide is a single-stranded component of a scaffold adapter. A polynucleotide herein generally refers to a single-stranded multimer of nucleotide from 5 to 500 nucleotides, e.g., 5 to 100 nucleotides. Polynucleotides may be synthetic or may be made enzymatically, and, in some embodiments, are about 5 to 50 nucleotides in length. Polynucleotides may contain ribonucleotide monomers (i.e., may be polyribonucleotides or “RNA polynucleotides”), deoxyribonucleotide monomers (i.e., may be polydeoxyribonucleotides or “DNA polynucleotides”), or a combination thereof. Polynucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example. The terms polynucleotide and oligonucleotide may be used interchangeably.

A scaffold polynucleotide may include an ssNA hybridization region (also referred to as scaffold, scaffold region, single-stranded scaffold, single-stranded scaffold region) and an oligonucleotide hybridization region. An ssNA hybridization region and an oligonucleotide hybridization region may be referred to as subcomponents of a scaffold polynucleotide. An ssNA hybridization region typically comprises a polynucleotide that hybridizes, or is capable of hybridizing, to an ssNA terminal region. An oligonucleotide hybridization region typically comprises a polynucleotide that hybridizes, or is capable of hybridizing, to all or a portion of the oligonucleotide component of the scaffold adapter.

An ssNA hybridization region of a scaffold polynucleotide may comprise a polynucleotide that is complementary, or substantially complementary, to an ssNA terminal region. In some embodiments, an ssNA hybridization region comprises a random sequence. In some embodiments, an ssNA hybridization region comprises a sequence complementary to an ssNA terminal region sequence of interest (e.g., targeted sequence). In certain embodiments, an ssNA hybridization region comprises one or more nucleotides that are all capable of non-specific base pairing to bases in the ssNA. Nucleotides capable of non-specific base pairing may be referred to as universal bases. A universal base is a base capable of indiscriminately base pairing with each of the four standard nucleotide bases: A, C, G and T. Universal bases that may be incorporated into the ssNA hybridization region include, but are not limited to, inosine, deoxyinosine, 2′-deoxyinosine (dl, dlnosine), nitroindole, 5-nitroindole, and 3-nitropyrrole. In certain embodiments, an ssNA hybridization region comprises one or more degenerate/wobble bases which can replace two or three (but not all) of the four typical bases (e.g., non-natural base P and K).

An ssNA hybridization region of a scaffold polynucleotide may have any suitable length and sequence. In some embodiments, the length of the ssNA hybridization region is 10 nucleotides or less. In certain aspects, the ssNA hybridization region is from 4 to 100 nucleotides in length, e.g., about 5, 10, 15, 20, 25, 30, 35, 40, 45, 50, 55, 60, 65, 70, 75, 80, 85, 90, 95, or 100 nucleotides in length. In certain aspects, the ssNA hybridization region is from 4 to 20 nucleotides in length, e.g., from 5 to 15, 5 to 10, 5 to 9, 5 to 8, or 5 to 7 (e.g., 6 or 7) nucleotides in length. In some embodiments, the ssNA hybridization region is 7 nucleotides in length. In some embodiments, the ssNA hybridization region comprises or consists of a random nucleotide sequence, such that when a plurality of heterogeneous scaffold polynucleotides having various random ssNA hybridization regions are employed, the collection is capable of acting as scaffold polynucleotides for a heterogeneous population of ssNAs irrespective of the sequences of the terminal regions of the ssNAs. Each scaffold polynucleotide having a unique ssNA hybridization region sequence may be referred to as a scaffold polynucleotide species and a collection of multiple scaffold polynucleotide species may be referred to as a plurality of scaffold polynucleotide species (e.g., for a scaffold polynucleotide designed to have 7 random bases in the ssNA hybridization region, a plurality of scaffold polynucleotide species would include 4⁷ unique ssNA hybridization region sequences). Accordingly, each scaffold adapter having a unique scaffold polynucleotide (i.e., comprising a unique ssNA hybridization region sequence) may be referred to as a scaffold adapter species and a collection of multiple scaffold adapter species may be referred to as a plurality of scaffold adapter species. A species of scaffold polynucleotide generally contains a feature that is unique with respect to other scaffold polynucleotide species. For example, a scaffold polynucleotide species may contain a unique sequence feature. A unique sequence feature may include a unique sequence length, a unique nucleotide sequence (e.g., a unique random sequence, a unique targeted sequence), or a combination of a unique sequence length and nucleotide sequence.

An oligonucleotide is a further single-stranded component of a scaffold adapter. An oligonucleotide herein generally refers to a single-stranded multimer of nucleotides from 5 to 500 nucleotides, e.g., 5 to 100 nucleotides. Oligonucleotides may be synthetic or may be made enzymatically, and, in some embodiments, are 5 to 50 nucleotides in length. Oligonucleotides may contain ribonucleotide monomers (i.e., may be oligoribonucleotides or “RNA oligonucleotides”), deoxyribonucleotide monomers (i.e., may be oligodeoxyribonucleotides or “DNA oligonucleotides”), or a combination thereof. Oligonucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example. The terms oligonucleotide and polynucleotide may be used interchangeably.

An oligonucleotide component of a scaffold adapter generally comprises a nucleic acid sequence that is complementary or substantially complementary to the oligonucleotide hybridization region of the scaffold polynucleotide. An oligonucleotide component of a scaffold adapter may include one or more subcomponents useful for one or more downstream applications such as, for example, PCR amplification of the ssNA fragment or derivative thereof, sequencing of the ssNA or derivative thereof, and the like. In some embodiments, a subcomponent of an oligonucleotide is a sequencing adapter. Sequencing adapter generally refers to one or more nucleic acid domains that include at least a portion of a nucleotide sequence (or complement thereof) utilized by a sequencing platform of interest, such as a sequencing platform provided by Illumina® (e.g., the HiSeq™, MiSeq™ and/or Genome Analyzer™ sequencing systems); Oxford Nanopore™ Technologies (e.g., the MinION™ sequencing system), Ion Torrent™ (e.g., the Ion PGM™ and/or Ion Proton™ sequencing systems); Pacific Biosciences (e.g., a Sequel or PACBIO RS II sequencing system); Life Technologies™ (e.g., a SOLiD™ sequencing system); Roche (e.g., the 454 GS FLX+ and/or GS Junior sequencing systems); or any sequencing platform of interest.

In some embodiments, an oligonucleotide component of a scaffold adapter is, or comprises, a nucleic acid domain selected from: a domain (e.g., a “capture site” or “capture sequence”) that specifically binds to a surface-attached sequencing platform oligonucleotide (e.g., a P5 or P7 oligonucleotide attached to the surface of a flow cell in an Illumina® sequencing system); a sequencing primer binding domain (e.g., a domain to which the Read 1 or Read 2 primers of the Illumina® platform may bind); a unique identifier or index (e.g., a barcode or other domain that uniquely identifies the sample source of the ssNA being sequenced to enable sample multiplexing by marking every molecule from a given sample with a specific barcode or “tag”); a barcode sequencing primer binding domain (a domain to which a primer used for sequencing a barcode binds); a molecular identification domain or unique molecular identifier (UMI) (e.g., a molecular index tag, such as a randomized tag of 4, 6, or other number of nucleotides) for uniquely marking molecules of interest, e.g., to determine expression levels based on the number of instances a unique tag is sequenced; a complement of any such domains; or any combination thereof. In some embodiments, a barcode domain (e.g., sample index tag) and a molecular identification domain (e.g., a molecular index tag; UMI) may be included in the same nucleic acid.

When an oligonucleotide component of a scaffold adapter includes one or a portion of a sequencing adapter, one or more additional sequencing adapters and/or a remaining portion of the sequencing adapter may be added using a variety of approaches. For example, additional and/or remaining portions of sequencing adapters may be added by any one of ligation, reverse transcription, PCR amplification, and the like. In the case of PCR, an amplification primer pair may be employed that includes a first amplification primer that includes a 3′ hybridization region (e.g., for hybridizing to an adapter region of the oligonucleotide) and a 5′ region including an additional and/or remaining portion of a sequencing adapter, and a second amplification primer that includes a 3′ hybridization region (e.g., for hybridizing to an adapter region of a second oligonucleotide added to the opposite end of an ssNA molecule) and optionally a 5′ region including an additional and/or remaining portion of a sequencing adapter.

The scaffold polynucleotide may be hybridized to the oligonucleotide, forming a duplex in the scaffold adapter. Accordingly, a scaffold adapter may be referred to as a scaffold duplex, a duplex adapter, a duplex oligonucleotide, or a duplex polynucleotide. Each scaffold duplex having a unique scaffold polynucleotide (i.e., comprising a unique ssNA hybridization region sequence) may be referred to as a scaffold duplex species and a collection of multiple scaffold duplex species may be referred to as a plurality of scaffold duplex species. In some embodiments, the scaffold polynucleotide and the oligonucleotide are on separate DNA strands. In some embodiments, the scaffold polynucleotide and the oligonucleotide are on a single DNA strand (e.g., a single DNA strand capable of forming a hairpin structure).

A method herein may comprise combining one or more scaffold adapters, or components thereof, with a composition comprising single-stranded nucleic acid (ssNA) to form one or more complexes. The scaffold polynucleotide is designed for simultaneous hybridization to an ssNA fragment and an oligonucleotide component such that, upon complex formation, an end of the oligonucleotide component is adjacent to an end of the terminal region of the ssNA fragment. Typically, upon complex formation, a 5′ end of the oligonucleotide component is adjacent to a 3′ end of the terminal region of the ssNA, or a 5′ end of the oligonucleotide component is adjacent to a 3′ end of the terminal region of the ssNA. Upon complex formation in instances where a scaffold adapter is attached to both ends of an ssNA fragment, a 5′ end of one oligonucleotide component is adjacent to a 3′ end of one terminal region of the ssNA, and a 5′ end of a second oligonucleotide component is adjacent to a 3′ end of a second terminal region of the ssNA.

In some embodiments, a method includes forming complexes by combining an ssNA composition, an oligonucleotide, and a plurality of heterogeneous scaffold polynucleotides having various random ssNA hybridization regions capable of acting as scaffolds for a heterogeneous population of ssNA having terminal regions of undetermined sequence.

In some embodiments, an ssNA hybridization region includes a known sequence designed to hybridize to an ssNA terminal region of known sequence. In some embodiments, two or more heterogeneous scaffold polynucleotides having different ssNA hybridization regions of known sequence are designed to hybridize to respective ssNA terminal regions of known sequence. Embodiments in which the ssNA hybridization regions have a known sequence may be useful, for example, for producing a nucleic acid library from a subset of ssNAs having terminal regions of known sequence. Accordingly, in certain embodiments, a method herein comprises forming complexes by combining an ssNA composition, an oligonucleotide, and one or more heterogeneous scaffold polynucleotides having one or more different ssNA hybridization regions of known sequence capable of acting as scaffolds for one or more ssNAs having one or more terminal regions of known sequence.

An ssNA fragment, an oligonucleotide, and scaffold polynucleotide may be combined in various ways. In some configurations, the combining includes combining 1) a complex comprising the scaffold polynucleotide hybridized to the oligonucleotide component via the oligonucleotide hybridization region, and 2) the ssNA fragment. In another configuration, the combining includes combining 1) a complex comprising the scaffold polynucleotide hybridized to the ssNA fragment via the ssNA hybridization region, and 2) the oligonucleotide component. In another configuration, the combining includes combining 1) the ssNA fragment, 2) the oligonucleotide, and 3) the scaffold polynucleotide, where none of the three components are pre-complexed with, or hybridized to, another component prior to the combining.

The combining may be carried out under hybridization conditions such that complexes form including a scaffold polynucleotide hybridized to a terminal region of an ssNA fragment via the ssNA hybridization region, and the scaffold polynucleotide hybridized to an oligonucleotide component via the oligonucleotide hybridization region. Whether specific hybridization occurs may be determined by factors such as the degree of complementarity between the hybridizing regions of the scaffold polynucleotide, the terminal region of the ssNA fragment, and the oligonucleotide component, as well as the length thereof, salt concentration, and the temperature at which the hybridization occurs, which may be informed by the melting temperatures (Tm) of the relevant regions.

Complexes may be formed such that an end of an oligonucleotide component is adjacent to an end of a terminal region of an ssNA fragment. Adjacent to refers the terminal nucleotide at the end of the oligonucleotide and the terminal nucleotide end of the terminal region of the ssNA fragment are sufficiently proximal to each other that the terminal nucleotides may be covalently linked, for example, by chemical ligation, enzymatic ligation, or the like. In some embodiments, the ends are adjacent to each other by virtue of the terminal nucleotide at the end of the oligonucleotide and the terminal nucleotide end of the terminal region of the ssNA being hybridized to adjacent nucleotides of the scaffold polynucleotide. The scaffold polynucleotide may be designed to ensure that an end of the oligonucleotide is adjacent to an end of the terminal region of the ssNA fragment.

Nucleic acid fragments (e.g., ssNA fragments) may be combined with scaffold adapters, or components thereof, thereby generating combined products. In some embodiments, a method herein comprises contacting ssNA with single-stranded nucleic acid binding protein (SSB) to produce SSB-bound ssNA prior to or during combining with scaffold adapters, or components thereof. SSB generally binds in a cooperative manner to ssNA and typically does not bind well to double-stranded nucleic acid (dsNA). Upon binding ssDNA, SSB destabilizes helical duplexes. SSBs may be prokaryotic SSB (e.g., bacterial or archaeal SSB) or eukaryotic SSB. Examples of SSBs may include E. coli SSB, E. coli RecA, Extreme Thermostable Single-Stranded DNA Binding Protein (ET SSB), Thermus thermophilus (Tth) RecA, T4 Gene 32 Protein, replication protein A (RPA—a eukaryotic SSB), and the like. ET SSB, Tth RecA, E. coli RecA, T4 Gene 32 Protein, as well buffers and detailed protocols for preparing SSB-bound ssNA using such SSBs are commercially available (e.g., New England Biolabs, Inc. (Ipswich, Mass.)).

Combining ssNA fragments with scaffold adapters, or components thereof, may comprise hybridization and/or ligation (e.g., ligation of hybridization products). A combined product may include an ssNA fragment connected to (e.g., hybridized to and/or ligated to) a scaffold adapter, or component thereof, at one or both ends of the ssNA fragment. A combined product may include an ssNA fragment hybridized to a scaffold adapter, or component thereof, at one or both ends of the ssNA fragment, which may be referred to as a hybridization product. A combined product may include an ssNA fragment ligated to a scaffold adapter, or component thereof, at one or both ends of the ssNA fragment, which may be referred to as a ligation product. In some embodiments, products from a cleavage step (i.e., cleaved products) may be combined with scaffold adapters, or components thereof, thereby generating combined products. Certain methods herein comprise generating sets of combined products (e.g., a first set of combined products and a second set of combined products). In some embodiments, a first set of combined products includes ssNAs connected to (e.g., hybridized to and/or ligated to) scaffold adapters, or components thereof, from a first set of scaffold adapters, or components thereof. In some embodiments, a second set of combined products includes the first set of combined products connected to (e.g., hybridized to and/or ligated to) scaffold adapters, or components thereof, from a second set of scaffold adapters, or components thereof.

ssNAs may be combined with scaffold adapters, or components thereof, under hybridization conditions, thereby generating hybridization products. In some embodiments, the scaffold adapters are provided as pre-hybridized products and the hybridization step includes hybridizing the scaffold adapters to the ssNA. In some embodiments, the scaffold adapter components (i.e., oligonucleotides and scaffold polynucleotides) are provided as individual components and the hybridization step includes hybridizing the scaffold adapter components 1) to each other and 2) to the ssNA. In some embodiments, the scaffold adapter components (i.e., oligonucleotides and scaffold polynucleotides) are provided sequentially as individual components and the hybridization steps includes 1) hybridizing the scaffold polynucleotides to the ssNA, and then 2) hybridizing the oligonucleotides to the oligonucleotide hybridization region of the scaffold polynucleotides. The conditions during the combining step are those conditions in which scaffold adapters, or components thereof (e.g., single-stranded scaffold regions), specifically hybridize to ssNAs having a terminal region or terminal regions that are complementary in sequence with respect to the single-stranded scaffold regions. The conditions during the combining step also may include those conditions in which components of the scaffold adapters (e.g., oligonucleotides and oligonucleotide hybridization regions within the scaffold polynucleotides), specifically hybridize, or remain hybridized, to each other.

Specific hybridization may be affected or influenced by factors such as the degree of complementarity between the single-stranded scaffold regions and the ssNA terminal region(s), or between the oligonucleotides and oligonucleotide hybridization regions, the length thereof, and the temperature at which the hybridization occurs, which may be informed by melting temperatures (Tm) of the single-stranded scaffold regions. Melting temperature generally refers to the temperature at which half of the single-stranded scaffold regions/ssNA terminal regions remain hybridized and half of the single-stranded scaffold regions/ssNA terminal regions dissociate into single strands. The Tm of a duplex may be experimentally determined or predicted using the following formula Tm=81.5+16.6(log₁₀[Na+])+0.41 (fraction G+C)−(60/N), where N is the chain length and [Na+] is less than 1 M. Additional models that depend on various parameters also may be used to predict Tm of relevant regions depending on various hybridization conditions. Approaches for achieving specific nucleic acid hybridization are described, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

In some embodiments, a method herein comprises exposing hybridization products to conditions under which an end of an ssNA is joined to an end of a scaffold adapter to which it is hybridized. In particular, a method herein may comprise exposing hybridization products to conditions under which an end of an ssNA is joined to an end of an oligonucleotide component of a scaffold adapter to which it is hybridized. Joining may be achieved by any suitable approach that permits covalent attachment of ssNA to the scaffold adapter and/or oligonucleotide component of a scaffold adapter to which it is hybridized. When one end of an ssNA is joined to an end of a scaffold adapter and/or oligonucleotide component of a scaffold adapter to which it is hybridized, typically one of two attachment events is conducted: 1) the 3′ end of the ssNA to the 5′ end of the oligonucleotide component of the scaffold adapter, or 2) the 5′ end of the ssNA to the 3′ end of the oligonucleotide component of the scaffold adapter. When both ends of an ssNA are each joined to an end of a scaffold adapter and/or oligonucleotide component of a scaffold adapter to which it is hybridized, typically two attachment events are conducted: 1) the 3′ end of the ssNA to the 5′ end of the oligonucleotide component of a first scaffold adapter, and 2) the 5′ end of the ssNA to the 3′ end of the oligonucleotide component of a second scaffold adapter.

In some embodiments, a method herein comprises contacting hybridization products with an agent comprising a ligase activity under conditions in which an end of an ssNA is covalently linked to an end of a scaffold adapter and/or oligonucleotide component of a scaffold adapter to which the target nucleic acid (ssNA) is hybridized. Ligase activity may include, for example, blunt-end ligase activity, nick-sealing ligase activity, sticky end ligase activity, circularization ligase activity, cohesive end ligase activity, DNA ligase activity, RNA ligase activity, single-stranded ligase activity, and double-stranded ligase activity. Ligase activity may include ligating a 5′ phosphorylated end of one polynucleotide to a 3′ OH end of another polynucleotide (5′P to 3′OH). Ligase activity may include ligating a 3′ phosphorylated end of one polynucleotide to a 5′ OH end of another polynucleotide (3′P to 5′OH). Ligase activity may include ligating a 5′ end of an ssNA to a 3′ end of a scaffold adapter and/or oligonucleotide component of a scaffold adapter hybridized thereto in a ligation reaction. Ligase activity may include ligating a 3′ end of an ssNA to a 5′ end of a scaffold adapter and/or oligonucleotide component of a scaffold adapter hybridized thereto in a ligation reaction. Suitable reagents (e.g., ligases) and kits for performing ligation reactions are known and available. For example, Instant Sticky-end Ligase Master Mix available from New England Biolabs (Ipswich, Mass.) may be used. Ligases that may be used include, for example, T4 DNA ligase (e.g., at low or high concentration), T7 DNA Ligase, E. coli DNA Ligase, Electro Ligase®, RNA ligases, T4 RNA ligase 2, SplintR® Ligase, RtcB ligase, and the like and combinations thereof. When needed, a phosphate group may be added at the 5′ end of the oligonucleotide component or ssNA fragment using a suitable kinase, for example, such as T4 polynucleotide kinase (PNK). Such kinases and guidance for using such kinases to phosphorylate 5′ ends are available, for example, from New England BioLabs, Inc. (Ipswich, Mass.).

In some embodiments, a method comprises covalently linking the adjacent ends of an oligonucleotide component and an ssNA terminal region, thereby generating covalently linked hybridization products. In some embodiments, the covalently linking comprises contacting the hybridization products (e.g., ssNA fragments hybridized to at least one scaffold adapter herein) with an agent comprising a ligase activity under conditions in which the end of an ssNA terminal region is covalently linked to an end of the oligonucleotide component. In some embodiments, a method comprises covalently linking the adjacent ends of a first oligonucleotide component and a first ssNA terminal region, and covalently linking the adjacent ends of a second oligonucleotide component and a second ssNA terminal region, thereby generating covalently linked hybridization products. In some embodiments, the covalently linking comprises contacting hybridization products (e.g., ssNA fragments each hybridized two scaffold adapters herein) with an agent comprising a ligase activity under conditions in which an end of a first ssNA terminal region is covalently linked to an end of a first oligonucleotide component and an end of a second ssNA terminal region is covalently linked to an end of a second oligonucleotide component. In some embodiments, the agent comprising a ligase activity is a T4 DNA ligase.

In some embodiments, hybridization products are contacted with a first agent comprising a first ligase activity and a second agent comprising a second ligase activity different than the first ligase activity. For example, the first ligase activity and the second ligase activity independently may be chosen from blunt-end ligase activity, nick-sealing ligase activity, sticky end ligase activity, circularization ligase activity, and cohesive end ligase activity, double-stranded ligase activity, single-stranded ligase activity, 5′P to 3′OH ligase activity, and 3′P to 5′OH ligase activity.

Covalently linking the adjacent ends of an oligonucleotide and an ssNA fragment produces a covalently linked product, which may be referred to a ligation product. A covalently linked product that includes an ssNA fragment covalently linked to an oligonucleotide component, which remain hybridized to a scaffold polynucleotide, may be referred to as a covalently linked hybridization product. A covalently linked hybridization product may be denatured (e.g., heat-denatured) to separate the ssNA fragment covalently linked to an oligonucleotide component from the scaffold polynucleotide. A covalently linked product that includes an ssNA fragment covalently linked to an oligonucleotide component, which is no longer hybridized to a scaffold polynucleotide (e.g., after denaturing), may be referred to as a single-stranded ligation product. In some cases, portions of a scaffold polynucleotide can be cleaved and/or degraded, for example by using uracil-DNA glycosylase and an endonuclease at one or more uracil bases in the scaffold polynucleotide.

A covalently linked hybridization product and/or single-stranded ligation product may be purified prior to use as input in a downstream application of interest (e.g., amplification; sequencing). For example, covalently linked hybridization products and/or single-stranded ligation products may be purified from certain components present during the combining, hybridization, and/or covalently linking (ligation) steps (e.g., by solid phase reversible immobilization (SPRI), column purification, and/or the like).

Sequencing adapters may comprise sequences complementary to flow-cell anchors, and sometimes are utilized to immobilize a nucleic acid library to a solid support, such as the inside surface of a flow cell, for example. In some embodiments, a sequencing adapter comprises an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, a sequencing adapter comprises one or more of primer annealing polynucleotide, also referred to herein as priming sequence or primer binding domain, (e.g., for annealing to flow cell attached oligonucleotides and/or to free amplification primers), an index polynucleotide (e.g., sample index sequence for tracking nucleic acid from different samples; also referred to as a sample ID), a barcode polynucleotide (e.g., single molecule barcode (SMB) for tracking individual molecules of sample nucleic acid that are amplified prior to sequencing; also referred to as a molecular barcode or a unique molecular identifier (UMI)). In some embodiments, a primer annealing component (or priming sequence or primer binding domain) of a sequencing adapter comprises one or more universal sequences (e.g., sequences complementary to one or more universal amplification primers). In some embodiments, an index polynucleotide (e.g., sample index; sample ID) is a component of a sequencing adapter. In some embodiments, an index polynucleotide (e.g., sample index; sample ID) is a component of a universal amplification primer sequence.

In some embodiments, sequencing adapters when used in combination with amplification primers (e.g., universal amplification primers) are designed generate library constructs comprising one or more of: universal sequences, molecular barcodes, sample ID sequences, spacer sequences, and a sample nucleic acid sequence. In some embodiments, sequencing adapters when used in combination with universal amplification primers are designed to generate library constructs comprising an ordered combination of one or more of: universal sequences, molecular barcodes, sample ID sequences, spacer sequences, and a sample nucleic acid sequence. For example, a library construct may comprise a first universal sequence, followed by a second universal sequence, followed by first molecular barcode, followed by a spacer sequence, followed by a template sequence (e.g., sample nucleic acid sequence), followed by a spacer sequence, followed by a second molecular barcode, followed by a third universal sequence, followed by a sample ID, followed by a fourth universal sequence. In some embodiments, sequencing adapters when used in combination with amplification primers (e.g., universal amplification primers) are designed generate library constructs for each strand of a template molecule (e.g., sample nucleic acid molecule). In some embodiments, sequencing adapters are duplex adapters.

An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). In some embodiments, an identifier is incorporated into or attached to a nucleic acid prior to a sequencing method (e.g., by an extension reaction, by an amplification reaction, by a ligation reaction). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments, an identifier, a sequencing-specific index/barcode, and a sequencer-specific flow-cell binding primer sites are incorporated into a nucleic acid library by single-primer extension (e.g., by a strand displacing polymerase).

In some embodiments, a nucleic acid library or parts thereof are amplified (e.g., amplified by a PCR-based method) under amplification conditions. In some embodiments, a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments, solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WILDFIRE amplification (e.g., U.S. Patent Application Publication No. 2013/0012399), the like or combinations thereof.

Nucleic Acid Sequencing

In some embodiments, nucleic acid (e.g., nucleic acid fragments, target nucleic acid, sample nucleic acid, cell-free nucleic acid, double-stranded nucleic acid, double-stranded DNA, single-stranded nucleic acid, single-stranded DNA, single-stranded RNA) is sequenced. In some embodiments, nucleic acids hybridized to sequencing adapters (“hybridization products”) are sequenced by a sequencing process. In some embodiments, nucleic acids ligated to sequencing adapters (“ligation products”) are sequenced by a sequencing process. In some embodiments, hybridization products and/or ligation products are amplified by an amplification process, and the amplification products are sequenced by a sequencing process. In some embodiments, hybridization products and/or ligation products are not amplified by an amplification process, and the hybridization products and/or ligation products are sequenced without prior amplification by a sequencing process. In some embodiments, the sequencing process generates sequence reads (or sequencing reads). In some embodiments, a method herein comprises determining the sequence of a nucleic acid molecule based on the sequence reads.

For certain sequencing platforms (e.g., paired-end sequencing), generating sequence reads may include generating forward sequence reads and generating reverse sequence reads. For example, sequencing using certain paired-end sequencing platforms sequence each nucleic acid fragment from both directions, generally resulting in two reads per nucleic acid fragment, with the first read in a forward orientation (forward read) and the second read in reverse-complement orientation (reverse read). For certain platforms, a forward read is generated off a particular primer within a sequencing adapter (e.g., ILLUMINA adapter, P5 primer), and a reverse read is generated off a different primer within a sequencing adapter (e.g., ILLUMINA adapter, P7 primer).

Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MINION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides.

The length of a sequence read is often associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp.

In some embodiments. the nominal, average, mean or absolute length of single-end reads sometimes is about 10 continuous nucleotides to about 250 or more contiguous nucleotides, about 15 contiguous nucleotides to about 200 or more contiguous nucleotides, about 15 contiguous nucleotides to about 150 or more contiguous nucleotides, about 15 contiguous nucleotides to about 125 or more contiguous nucleotides, about 15 contiguous nucleotides to about 100 or more contiguous nucleotides, about 15 contiguous nucleotides to about 75 or more contiguous nucleotides, about 15 contiguous nucleotides to about 60 or more contiguous nucleotides, 15 contiguous nucleotides to about 50 or more contiguous nucleotides, about 15 contiguous nucleotides to about 40 or more contiguous nucleotides, and sometimes about 15 contiguous nucleotides or about 36 or more contiguous nucleotides. In certain embodiments the nominal, average, mean or absolute length of single-end reads is about 20 to about 30 bases, or about 24 to about 28 bases in length. In certain embodiments the nominal, average, mean or absolute length of single-end reads is about 1, 2, 3, 4, 5, 6, 7, 8, 9, 10, 11, 12, 13, 14, 15, 16, 17, 18, 19, 21, 22, 23, 24, 25, 26, 27, 28 or about 29 bases or more in length. In certain embodiments the nominal, average, mean or absolute length of single-end reads is about 20 to about 200 bases, about 100 to about 200 bases, or about 140 to about 160 bases in length. In certain embodiments the nominal, average, mean or absolute length of single-end reads is about 30, 40, 50, 60, 70, 80, 90, 100, 110, 120, 130, 140, 150, 160, 170, 180, 190, or about 200 bases or more in length. In certain embodiments, the nominal, average, mean or absolute length of paired-end reads sometimes is about 10 contiguous nucleotides to about 25 contiguous nucleotides or more (e.g., about 10, 11, 12, 13, 14, 15, 16, 17, 18, 19, 20, 21, 22, 23, 24 or 25 nucleotides in length or more), about 15 contiguous nucleotides to about 20 contiguous nucleotides or more, and sometimes is about 17 contiguous nucleotides or about 18 contiguous nucleotides. In certain embodiments, the nominal, average, mean or absolute length of paired-end reads sometimes is about 25 contiguous nucleotides to about 400 contiguous nucleotides or more (e.g., about 25, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nucleotides in length or more), about 50 contiguous nucleotides to about 350 contiguous nucleotides or more, about 100 contiguous nucleotides to about 325 contiguous nucleotides, about 150 contiguous nucleotides to about 325 contiguous nucleotides, about 200 contiguous nucleotides to about 325 contiguous nucleotides, about 275 contiguous nucleotides to about 310 contiguous nucleotides, about 100 contiguous nucleotides to about 200 contiguous nucleotides, about 100 contiguous nucleotides to about 175 contiguous nucleotides, about 125 contiguous nucleotides to about 175 contiguous nucleotides, and sometimes is about 140 contiguous nucleotides to about 160 contiguous nucleotides. In certain embodiments, the nominal, average, mean, or absolute length of paired-end reads is about 150 contiguous nucleotides, and sometimes is 150 contiguous nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from a sample from a subject can be reads from a mixture of a minority nucleic acid and a majority nucleic acid. For example, sequence reads obtained from the blood of a cancer patient can be reads from a mixture of cancer nucleic acid and non-cancer nucleic acid. In another example, sequence reads obtained from the blood of a pregnant female can be reads from a mixture of fetal nucleic acid and maternal nucleic acid. In another example, sequence reads obtained from the blood of a patient having an infection or infectious disease can be reads from a mixture of host nucleic acid and pathogen nucleic acid. In another example, sequence reads obtained from the blood of a transplant recipient can be reads from a mixture of host nucleic acid and transplant nucleic acid. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces) in a subject. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces), and nucleic acid from the host subject. A mixture of relatively short reads can be transformed by processes described herein into a representation of genomic nucleic acid present in the subject, and/or a representation of genomic nucleic acid present in a tumor, a fetus, a pathogen, a transplant, or a microbiome.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments, specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, a genome (e.g., a whole genome) is sequenced with about 1-fold to about 200-fold coverage, or about 50-fold to 100-fold coverage (e.g., about 1-, 2-, 3-, 4-, 5-, 6-, 7-, 8-, 9-, 10-, 20-, 30-, 40-, 50-, 60-, 70-, 80-, 90-, 100-, 150-, 200-fold or greater coverage). In some embodiments, a genome (e.g., a whole genome) is sequenced with at least about 1-fold coverage. In some embodiments, a genome (e.g., a whole genome) is sequenced with at least about 2-fold coverage. In some embodiments, a genome (e.g., a whole genome) is sequenced with about 10-fold coverage. In some embodiments, a genome (e.g., a whole genome) is sequenced with about 50-fold coverage. In some embodiments, a genome (e.g., a whole genome) is sequenced with about 100-fold coverage.

In some embodiments, a test sample is sequenced using low coverage sequencing. Low coverage sequencing may be referred to as shallow depth sequencing. Low coverage sequencing may refer to sequencing at about 10-fold coverage or less. In some embodiments, a test sample is sequenced at about 10-fold coverage or less. In some embodiments, a test sample is sequenced at about 9-fold coverage or less. In some embodiments, a test sample is sequenced at about 8-fold coverage or less. In some embodiments, a test sample is sequenced at about 7-fold coverage or less. In some embodiments, a test sample is sequenced at about 6-fold coverage or less. In some embodiments, a test sample is sequenced at about 5-fold coverage or less. In some embodiments, a test sample is sequenced at about 4-fold coverage or less. In some embodiments, a test sample is sequenced at about 3-fold coverage or less. In some embodiments, a test sample is sequenced at about 2-fold coverage or less. In some embodiments, a test sample is sequenced at about 1-fold coverage or less. In some embodiments, a test sample is sequenced at a fold coverage between about 0.5-fold to about 2-fold. In some embodiments, a test sample is sequenced at about 2-fold coverage. In some embodiments, a test sample is sequenced at about 1-fold coverage. In some embodiments, a test sample is sequenced at about 0.9-fold coverage or less. In some embodiments, a test sample is sequenced at about 0.8-fold coverage or less. In some embodiments, a test sample is sequenced at about 0.7-fold coverage or less. In some embodiments, a test sample is sequenced at about 0.6-fold coverage or less. In some embodiments, a test sample is sequenced at about 0.5-fold coverage or less.

In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used.

In some embodiments, a shotgun sequencing method is used. Shotgun sequencing generally refers to sequencing random nucleic acid strands. For example, DNA may be broken up randomly into numerous small fragments or DNA may be present as small fragments in a sample (e.g., cell-free DNA, degraded DNA). The DNA fragments are sequenced to obtain sequence reads. Multiple overlapping reads for the target DNA are obtained, and the overlapping reads are used to assemble the reads into a continuous sequence (typically performed using a computer program).

In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In certain embodiments, sequence reads are generated using a whole genome sequencing approach. In certain embodiments, sequence reads are generated using a genome-wide sequencing approach. In certain embodiments, sequence reads are generated using a massively parallel sequencing approach. In certain embodiments, sequence reads are generated by a non-targeted sequencing approach. In certain embodiments, sequence reads are generated using a genome-wide, massively parallel sequencing approach. In certain embodiments, sequence reads are generated using a non-targeted, genome-wide sequencing approach. In certain embodiments, sequence reads are generated using a non-targeted, massively parallel sequencing approach. In certain embodiments, sequence reads are generated using a non-targeted, genome-wide, massively parallel sequencing approach.

Whole genome, genome-wide, massively parallel, and/or non-targeted sequencing approaches generate massive amounts of data. The human genome is approximately 3 billion base pairs in size. An example sequencing process performed on a test sample at 1-fold coverage would generate at least 3 million 1 kb reads. Sequencing processes that produce smaller reads and/or are performed at greater than 1-fold coverage would generate more than 3 million reads. Accordingly, such sequence data typically is processed (e.g., aligned, analyzed for alleles at target and linked loci, quantified, assessed for genotypes) using a computer, as the sheer volume of such data makes it impractical or impossible for a human to perform such a task without the use of a computer and/or software. In some embodiments, a method herein comprises generating, obtaining, and/or processing at least 100,000 sequence reads. In some embodiments, a method herein comprises generating, obtaining, and/or processing at least 500,000 sequence reads. In some embodiments, a method herein comprises generating, obtaining, and/or processing at least 1,000,000 sequence reads. In some embodiments, a method herein comprises generating, obtaining, and/or processing at least 2,000,000 sequence reads. In some embodiments, a method herein comprises generating, obtaining, and/or processing at least 3,000,000 sequence reads.

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego Calif.)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include ILLUMINA/SOLEX/HISEQ (e.g., Illumina's Genome Analyzer; Genome Analyzer II; HISEQ 2000; HISEQ), SOLID, Roche/454, PACBIO and/or SMRT, Helicos True Single Molecule Sequencing, Ion Torrent and Ion semiconductor-based sequencing (e.g., as developed by Life Technologies), WILDFIRE, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LASERGEN systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, nucleic acid is sequenced and the sequencing product (e.g., a collection of sequence reads, sequence read data) is processed prior to, or in conjunction with, an analysis of the sequenced nucleic acid. For example, sequence reads and/or sequence read data may be processed according to one or more of the following: aligning, mapping, filtering, quantifying, generating genotype likelihoods, generating genotypes, performing a genealogy analysis, and the like, and combinations thereof. Certain processing steps may be performed in any order and certain processing steps may be repeated.

Aligning/Mapping Reads

Sequence reads can be aligned/mapped, and the number of reads carrying a particular allele or alleles are referred to as counts or quantifications (e.g., allele counts or allele quantifications). Any suitable aligning/mapping method (e.g., process, algorithm, program, software, module, the like or combination thereof) can be used. Certain aspects of aligning/mapping processes are described hereafter.

Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped,” as “a mapped sequence read” or as “a mapped read.”

The terms “aligned,” “alignment,” or “aligning” generally refer to two or more nucleic acid sequences that can be identified as a match (e.g., 100% identity) or partial match. Alignments are generally performed by a computer (e.g., a software, program, module, or algorithm), non-limiting examples of which include the Efficient Local Alignment of Nucleotide Data (ELAND) computer program distributed as part of the ILLUMINA Genomics Analysis pipeline. Alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments a nucleic acid sequence is aligned with the reverse complement of another nucleic acid sequence.

Various computational methods can be used to map each sequence read to a reference genome or portion thereof. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA (e.g., BWA-MEM aligner), or SEQMAP, or variations thereof or combinations thereof. In some embodiments, sequence reads are aligned with sequences in a reference genome. In some embodiments, sequence reads are found and/or aligned with sequences in nucleic acid databases known in the art including, for example, GENBANK, dbEST, dbSTS, EMBL (European Molecular Biology Laboratory) and DDBJ (DNA Databank of Japan). BLAST or similar tools can be used to search identified sequences against a sequence database.

In some embodiments, a read may uniquely or non-uniquely map to a reference genome. A read is considered as “uniquely mapped” if it aligns with a single sequence in the reference genome. A read is considered as “non-uniquely mapped” if it aligns with two or more sequences in the reference genome. In some embodiments, non-uniquely mapped reads are eliminated from further analysis (e.g. quantification). A certain, small degree of mismatch (0-1, 1 or more) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. For example, a reference genome used for human subjects as well as many other organisms can be found at the National Center for Biotechnology Information at World Wide Web URL ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, mappability is assessed for a genomic region (e.g., portion, genomic portion). Mappability is the ability to unambiguously align a nucleotide sequence read to a reference genome, or portion thereof, typically up to a specified number of mismatches, including, for example, 0, 1, 2 or more mismatches. For a given genomic region, the expected mappability can be estimated using a sliding-window approach of a preset read length and averaging the resulting read-level mappability values. Genomic regions comprising stretches of unique nucleotide sequence sometimes have a high mappability value.

For paired-end sequencing, reads may be mapped to a reference genome by use of a suitable mapping and/or alignment program or algorithm, non-limiting examples of which include BWA (Li H. and Durbin R. (2009) Bioinformatics 25, 1754-60), NOVOALIGN [Novocraft (2010)], Bowtie (Langmead B, et al., (2009) Genome Biol. 10:R25), SOAP2 (Li R, et al., (2009) Bioinformatics 25, 1966-67), BFAST (Homer N, et al., (2009) PLoS ONE 4, e7767), GASSST (Rizk, G. and Lavenier, D. (2010) Bioinformatics 26, 2534-2540), and MPSCAN (Rivals E., et al. (2009) Lecture Notes in Computer Science 5724, 246-260), and the like. Reads can be trimmed and/or merged by use of a suitable trimming and/or merging program or algorithm, non-limiting examples of which include Cutadapt, trimmomatic, SeqPrep, and usearch. Some paired-end reads, such as those from nucleic acid templates that are shorter than the sequencing read length, can have portions sequenced by both the forward read and the reverse read; in such cases, the forward and reverse reads can be merged into a single read using the overlap between the forward and reverse reads. Reads that do not overlap or that do not overlap sufficiently can remain unmerged and be mapped as paired reads. Paired-end reads may be mapped and/or aligned using a suitable short read alignment program or algorithm. Non-limiting examples of short read alignment programs include BarraCUDA, BFAST, BLASTN, BLAT, Bowtie, BWA, CASHX, CUDA-EC, CUSHAW, CUSHAW2, drFAST, ELAND, ERNE, GNUMAP, GEM, GensearchNGS, GMAP, Geneious Assembler, iSAAC, LAST, MAQ, mrFAST, mrsFAST, MOSAIK, MPscan, Novoalign, NovoalignCS, Novocraft, NextGENe, Omixon, PALMapper, Partek, PASS, PerM, QPalma, RazerS, REAL, cREAL, RMAP, rNA, RTG, Segemehl, SeqMap, Shrec, SHRiMP, SLIDER, SOAP, SOAP2, SOAP3, SOCS, SSAHA, SSAHA2, Stampy, SToRM, Subread, Subjunc, Taipan, UGENE, VelociMapper, TimeLogic, XpressAlign, ZOOM, the like or combinations thereof. Paired-end reads are often mapped to opposing ends of the same polynucleotide fragment, according to a reference genome. In some embodiments, read mates are mapped independently. In some embodiments, information from both sequence reads (i.e., from each end) is factored in the mapping process. A reference genome is often used to determine and/or infer the sequence of nucleic acids located between paired-end read mates. The term “discordant read pairs” as used herein refers to a paired-end read comprising a pair of read mates, where one or both read mates fail to unambiguously map to the same region of a reference genome defined, in part, by a segment of contiguous nucleotides. In some embodiments discordant read pairs are paired-end read mates that map to unexpected locations of a reference genome. Non-limiting examples of unexpected locations of a reference genome include (i) two different chromosomes, (ii) locations separated by more than a predetermined fragment size (e.g., more than 300 bp, more than 500 bp, more than 1000 bp, more than 5000 bp, or more than 10,000 bp), (iii) an orientation inconsistent with a reference sequence (e.g., opposite orientations), the like or a combination thereof. In some embodiments discordant read mates are identified according to a length (e.g., an average length, a predetermined fragment size) or expected length of template polynucleotide fragments in a sample. For example, read mates that map to a location that is separated by more than the average length or expected length of polynucleotide fragments in a sample are sometimes identified as discordant read pairs. Read pairs that map in opposite orientation are sometimes determined by taking the reverse complement of one of the reads and comparing the alignment of both reads using the same strand of a reference sequence. Discordant read pairs can be identified by any suitable method and/or algorithm known in the art or described herein (e.g., SVDetect, Lumpy, BreakDancer, BreakDancerMax, CREST, DELLY, the like or combinations thereof).

Classifications and Uses Thereof

Methods described herein can provide an outcome indicative of one or more characteristics of a sample or source described above. Methods described herein sometimes provide an outcome indicative of one or more genotypes for a test sample (e.g., providing an outcome determinative of one or more genotypes). Methods described herein sometimes provide an outcome indicative of an identification of a subject for a test sample (e.g., providing an outcome determinative of the identity of a subject). An outcome often is part of a classification process, and a classification (e.g., classification of one or more characteristics of a sample or source; and/or presence or absence of a genotype, phenotype, genetic variation, medical condition, and/or subject identification for a test sample) sometimes is based on and/or includes an outcome. An outcome and/or classification sometimes is based on and/or includes a result of data processing for a test sample that facilitates determining one or more characteristics of a sample or source and/or presence or absence of a genotype, phenotype, genetic variation, genetic alteration, medical condition, and/or subject identification in a classification process (e.g., a statistic value). An outcome and/or classification sometimes includes or is based on a score determinative of, or a call of, one or more characteristics of a sample or source and/or presence or absence of a genotype, phenotype, genetic variation, genetic alteration, medical condition, and/or subject identification. In certain embodiments, an outcome and/or classification includes a conclusion that predicts and/or determines one or more characteristics of a sample or source and/or presence or absence of a genotype, phenotype, genetic variation, genetic alteration, medical condition, and/or subject identification in a classification process.

Any suitable expression of an outcome and/or classification can be provided. An outcome and/or classification sometimes is based on and/or includes one or more numerical values generated using a processing method described herein in the context of one or more considerations of probability. Non-limiting examples of values that can be utilized include a sensitivity, specificity, standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, ploidy value, area ratio, median level, the like or combination thereof. In some embodiments, an outcome and/or classification comprises a genotype likelihood, a set of genotype likelihoods, a genotype likelihood ratio, and/or a set of genotype likelihood ratios. In certain embodiments, multiple values are analyzed together. A consideration of probability can facilitate determining one or more characteristics of a sample or source.

In certain embodiments, an outcome and/or classification is based on and/or includes a conclusion that predicts and/or determines a risk or probability of the presence or absence of a genotype, phenotype, genetic variation, medical condition, and/or subject identification for a test sample. A conclusion sometimes is based on a value determined from a data analysis method described herein (e.g., a statistics value indicative of probability, certainty and/or uncertainty (e.g., standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, sensitivity, specificity, the like or combination thereof). An outcome and/or classification sometimes is expressed in a laboratory test report for particular test sample as a probability (e.g., odds ratio, p-value), likelihood, or risk factor, associated with the presence or absence of a genotype, phenotype, genetic variation, medical condition, and/or subject identification. An outcome and/or classification for a test sample sometimes is provided as “positive” or “negative” with respect to a particular genotype, phenotype, genetic variation, medical condition, and/or subject identification. For example, an outcome and/or classification sometimes is designated as “positive” in a laboratory test report for a particular test sample where presence of a genotype, phenotype, genetic variation, medical condition, and/or subject identification is determined, and sometimes an outcome and/or classification is designated as “negative” in a laboratory test report for a particular test sample where absence of a genotype, phenotype, genetic variation, medical condition, and/or subject identification is determined. An outcome and/or classification sometimes is determined and sometimes includes an assumption used in data processing.

There typically are four types of classifications generated in a classification process: true positive, false positive, true negative and false negative. The term “true positive” as used herein refers to presence of a genotype, phenotype, genetic variation, medical condition, and/or subject identification correctly determined for a test sample. The term “false positive” as used herein refers to presence of a genotype, phenotype, genetic variation, medical condition, and/or subject identification incorrectly determined for a test sample. The term “true negative” as used herein refers to absence of a genotype, phenotype, genetic variation, medical condition, and/or subject identification correctly determined for a test sample. The term “false negative” as used herein refers to absence of a genotype, phenotype, genetic variation, medical condition, and/or subject identification incorrectly determined for a test sample. Two measures of performance for a classification process can be calculated based on the ratios of these occurrences: (i) a sensitivity value, which generally is the fraction of predicted positives that are correctly identified as being positives; and (ii) a specificity value, which generally is the fraction of predicted negatives correctly identified as being negative.

In certain embodiments, a laboratory test report generated for a classification process includes a measure of test performance (e.g., sensitivity and/or specificity) and/or a measure of confidence (e.g., a confidence level, confidence interval). A measure of test performance and/or confidence sometimes is obtained from a clinical validation study performed prior to performing a laboratory test for a test sample. In certain embodiments, one or more of sensitivity, specificity and/or confidence are expressed as a percentage. In some embodiments, a percentage expressed independently for each of sensitivity, specificity or confidence level, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). A confidence interval expressed for a particular confidence level (e.g., a confidence level of about 90% to about 99.9% (e.g., about 95%)) can be expressed as a range of values, and sometimes is expressed as a range or sensitivities and/or specificities for a particular confidence level. Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome and/or classification is not due to chance) in certain embodiments is expressed as a standard score (e.g., z-score), a p-value, or result of a t-test. In some embodiments, a measured variance, confidence level, confidence interval, sensitivity, specificity and the like (e.g., referred to collectively as confidence parameters) for an outcome and/or classification can be generated using one or more data processing manipulations described herein.

An outcome and/or classification for a test sample often is ordered by, and often is provided to, a health care professional, law enforcement professional, or other qualified individual who transmits an outcome and/or classification to a subject from whom the test sample is obtained. In certain embodiments, an outcome and/or classification is provided using a suitable visual medium (e.g., a peripheral or component of a machine, e.g., a printer or display). A classification and/or outcome often is provided to a healthcare professional, law enforcement professional, or qualified individual in the form of a report. A report typically comprises a display of an outcome and/or classification (e.g., a value, one or more characteristics of a sample or source, or an assessment or probability of presence or absence of a genotype, phenotype, genetic variation, medical condition, and/or subject identification), sometimes includes an associated confidence parameter, and sometimes includes a measure of performance for a test used to generate the outcome and/or classification. A report sometimes includes a recommendation for a follow-up procedure (e.g., a procedure that confirms the outcome or classification). A report sometimes includes a visual representation of a chromosome or portion thereof (e.g., a chromosome ideogram or karyogram), and sometimes shows a visualization of a duplication and/or deletion region for a chromosome (e.g., a visualization of a whole chromosome for a chromosome deletion or duplication; a visualization of a whole chromosome with a deleted region or duplicated region shown; a visualization of a portion of chromosome duplicated or deleted; a visualization of a portion of a chromosome remaining in the event of a deletion of a portion of a chromosome) identified for a test sample.

A report can be displayed in a suitable format that facilitates determination of presence or absence of a genotype, phenotype, genetic variation, medical condition, and/or subject identification by a health professional or other qualified individual. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing.

A report may be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, medical record file, or any other medium described in the previous paragraph. A laboratory file or medical record file may be in tangible form or electronic form (e.g., computer readable form), in certain embodiments. After a report is generated and transmitted, a report can be received by obtaining, via a suitable communication medium, a written and/or graphical representation comprising an outcome and/or classification, which upon review allows a healthcare professional, law enforcement professional, or other qualified individual to make a determination as to one or more characteristics of a sample or source, or presence or absence of a genotype, phenotype, genetic variation, medical condition, and/or subject identification for a test sample.

An outcome and/or classification may be provided by and obtained from a laboratory (e.g., obtained from a laboratory file). A laboratory file can be generated by a laboratory that carries out one or more tests for determining one or more characteristics of a sample or source and/or presence or absence of a genotype, phenotype, genetic variation, medical condition, and/or subject identification for a test sample. Laboratory personnel (e.g., a laboratory manager) can analyze information associated with test samples (e.g., test profiles, reference profiles, test values, reference values, level of deviation, patient information) underlying an outcome and/or classification. For calls pertaining to presence or absence of a genotype, phenotype, genetic variation, medical condition, and/or subject identification that are close or questionable, laboratory personnel can re-run the same procedure using the same (e.g., aliquot of the same sample) or different test sample from a test subject. A laboratory may be in the same location or different location (e.g., in another country) as personnel assessing the presence or absence of a genotype, phenotype, genetic variation, medical condition, and/or subject identification from the laboratory file. For example, a laboratory file can be generated in one location and transmitted to another location in which the information for a test sample therein is assessed by a healthcare professional, law enforcement professional, or other qualified individual, and optionally, transmitted to the subject from which the test sample was obtained. A laboratory sometimes generates and/or transmits a laboratory report containing a classification of presence or absence of genomic instability, a genotype, phenotype, a genetic variation, medical condition, and/or subject identification for a test sample. A laboratory generating a laboratory test report sometimes is a certified laboratory, and sometimes is a laboratory certified under the Clinical Laboratory Improvement Amendments (CLIA).

Machines, Software and Interfaces

Certain processes and methods described herein (e.g., selecting a subset of sequence reads, generating a sequence reads profile, processing sequence read data, processing sequence read quantifications, determining one or more characteristics of a sample based on sequence read data or a sequence read profile) often are too complex for performing in the mind and cannot be performed without a computer, microprocessor, software, module or other machine. Methods described herein may be computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., selecting a subset of sequence reads, generating a sequence reads profile, processing sequence read data, processing sequence read quantifications, determining one or more characteristics of a sample based on sequence read data or a sequence read profile) are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Machines, software and interfaces may be used to conduct methods described herein. Using machines, software and interfaces, a user may enter, request, query or determine options for using particular information, programs or processes (e.g., processing sequence read data, processing sequence read quantifications, and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read processing; send processed sequence read data to a computer system for further processing and/or yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine an outcome from the sequence reads (e.g., a characteristic of a sample).

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, sequence read information may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Sequence read quantifications (e.g., allele counts) sometimes are accessed by a microprocessor that executes instructions configured to carry out a method described herein. Sequence read quantifications that are accessed by a microprocessor can be within memory of a system, and the sequence read counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., IPADs, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining one or more characteristics of a sample.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

Provided herein, in certain embodiments, are systems, machines and apparatuses comprising one or more microprocessors and memory, which memory comprises instructions executable by the one or more microprocessors and which instructions executable by the one or more microprocessors are configured to generate allele quantifications and a set of genotype likelihoods, and, based on the set of genotype likelihoods, generate a genotype.

Provided herein, in certain embodiments, are machines comprising one or more microprocessors and memory, which memory comprises instructions executable by the one or more microprocessors and which memory comprises sequence reads aligned to a reference genome, and which instructions executable by the one or more microprocessors are configured to generate allele quantifications and a set of genotype likelihoods, and, based on the set of genotype likelihoods, generate a genotype.

Provided herein, in certain embodiments, are non-transitory computer-readable storage media with an executable program stored thereon, where the program instructs a microprocessor to perform the following: (a) access sequence reads aligned to a reference genome, (b) generate allele quantifications and a set of genotype likelihoods, and (c) based on the set of genotype likelihoods, generate a genotype.

Provided herein, in certain embodiments, are systems, machines and apparatuses comprising one or more microprocessors and memory, which memory comprises instructions executable by the one or more microprocessors and which instructions executable by the one or more microprocessors are configured to generate allele quantifications and a haplotype pair likelihood set, and, based on the haplotype pair likelihood set, generate a genotype.

Provided herein, in certain embodiments, are machines comprising one or more microprocessors and memory, which memory comprises instructions executable by the one or more microprocessors and which memory comprises sequence reads aligned to a reference genome, and which instructions executable by the one or more microprocessors are configured to generate allele quantifications and a haplotype pair likelihood set, and, based on the haplotype pair likelihood set, generate a genotype.

Provided herein, in certain embodiments, are non-transitory computer-readable storage media with an executable program stored thereon, where the program instructs a microprocessor to perform the following: (a) access sequence reads aligned to a reference genome, (b) generate allele quantifications and a haplotype pair likelihood set, and (c) based on the haplotype pair likelihood set, generate a genotype.

Kits

Provided in certain embodiments are kits. The kits may include any components and compositions described herein (e.g., sequencing adapters and components/subcomponents thereof, oligonucleotides, oligonucleotide components/regions, nucleic acids, primers, enzymes) useful for performing any of the methods described herein, in any suitable combination. Kits may further include any reagents, buffers, or other components useful for carrying out any of the methods described herein.

Kits may include components for capturing nucleic acid (e.g., cell free nucleic acid, damaged or degraded nucleic acid, fragmented nucleic acid) from a sample (e.g., a forensic sample). Kits for capturing nucleic acid from a forensic sample may be configured such that a user provides the sample nucleic acid.

Components of a kit may be present in separate containers, or multiple components may be present in a single container. Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for using sequencing adapters, or components thereof, to capture nucleic acid from a sample (e.g., a forensic sample) and/or to produce a nucleic acid library. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

Certain Implementations

Following are non-limiting examples of certain implementations of the technology.

A1. A method for generating a genotype for a target genomic locus for a test sample, comprising:
a) for a test sample comprising nucleic acid, obtaining sequence reads aligned to a reference genome;
b) from the sequence reads, quantifying a linked reference allele and quantifying a linked alternative allele, thereby generating allele quantifications for a linked genomic locus;
c) generating a set of genotype likelihoods for a target reference allele and a target alternative allele at the target genomic locus according to 1) a probability of a genotype at the target genomic locus based, in part, on the allele quantifications in (b), and 2) a probability of a genotype at the target genomic locus based on prior probabilities of the target reference allele and the target alternative allele; and
d) generating a genotype at the target genomic locus based on the set of genotype likelihoods.

A1.1 A method for generating a genotype for a target genomic locus for a test sample, comprising:
a) for a test sample comprising nucleic acid, obtaining sequence reads aligned to a reference genome;
b) from the sequence reads, quantifying a linked reference allele and quantifying a linked alternative allele, thereby generating allele quantifications for a linked genomic locus;
c) generating a set of genotype likelihoods for a target reference allele and a target alternative allele at the target genomic locus according to 1) a probability of the allele quantifications in (b), given a particular genotype at the target genomic locus, and 2) a probability of a genotype at the target genomic locus based on prior probabilities of the target reference allele and the target alternative allele; and
d) generating a genotype at the target genomic locus based on the set of genotype likelihoods.

A1.2 The method of embodiment A1 or A1.1, wherein the probability in (c)(1) is generated according to (i) a probability of observing the linked reference allele at the linked genomic locus, given a target reference allele at the target genomic locus, and/or (ii) a probability of observing the linked reference allele at the linked genomic locus, given a target alternative allele at the target genomic locus.

A1.3 The method of embodiment A1 or A1.1, wherein the probability in (c)(1) is generated according to (i) a probability of observing the linked reference allele at the linked genomic locus, given a target reference genotype at the target genomic locus, and/or (ii) a probability of observing the linked reference allele at the linked genomic locus, given a target alternative genotype at the target genomic locus.

A2. The method of embodiment A1.2 or A1.3, wherein the probability in (i) is based, in part, on a measure of linkage disequilibrium for the linked reference allele and the target reference allele.

A3. The method of embodiment A2, wherein the probability in (i) is adjusted according to a measure of sequencing error.

A4. The method of any one of embodiments A1.2 to A3, wherein the probability in (ii) is based, in part, on a measure of linkage disequilibrium for the linked reference allele and the target alternative allele.

A5. The method embodiment A4, wherein the probability in (ii) is adjusted according to a measure of sequencing error.

A6. The method of any one of embodiments A2 to A5, wherein the measure of disequilibrium is based on a haplotype frequency.

A7. The method of any one of embodiments A1 to A6, wherein the set of genotype likelihoods in (c) comprises one or more likelihoods for genotypes chosen from homozygous for the target reference allele, heterozygous for the target reference allele and the target alternative allele, and homozygous for the target alternative allele.

A8. The method of embodiment A7, wherein:
the probability of the homozygous for the target reference allele genotype in (c)(1) is generated according to a probability of observing the linked reference allele at the linked genomic locus, given a target reference allele at the target genomic locus;
the probability of the homozygous for the target alternative allele genotype in (c)(1) is generated according to a probability of observing the linked reference allele at the linked genomic locus, given a target alternative allele at the target genomic locus; and
the probability of the heterozygous for the target reference allele and the target alternative allele genotype in (c)(1) is generated according to one half the probability of observing the linked reference allele at the linked genomic locus, given a target reference allele at the target genomic locus, and one half the probability of observing the linked reference allele at the linked genomic locus, given a target alternative allele at the target genomic locus.

A9. The method of any one of embodiments A1 to A8, wherein the probability in (c)(2) is based, in part, on haplotype frequencies for (i) the target reference allele and the linked reference allele, (ii) the target reference allele and the linked alternative allele, (iii) the target alternative allele and the linked reference allele, and (iv) the target alternative allele and the linked alternative allele.

A10. The method of any one of embodiments A1 to A9, wherein (b) further comprises quantifying a target reference allele and quantifying a target alternative allele, thereby generating allele quantifications for the target genomic locus.

A11. The method of any one of embodiments A7 to A10, wherein the likelihood (L) for a homozygous target reference allele genotype (T00) is generated according to a process represented by equation (1):

$$L ( T 00 ) = P ( D \vert T 00 ) \times P ( T 00 ) = {\prod }_{{L}_{i}} ( \begin{matrix}{L}_{i} 0 + {L}_{i} 1 \\ {L}_{i} 0\end{matrix} ) \times {PL}_{i} {0}^{{L}_{i} 0} \times {( 1 - {PL}_{i} 0 )}^{{L}_{i} 1} \times {( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} )}^{2} \qquad ( 1 )$$

wherein
L(T00) is the likelihood of genotype 00 at target genomic locus T,
Lᵢ0 is the allele quantification of linked reference alleles observed at linked genomic locus Lᵢ,
Lᵢ1 is the allele quantification of linked alternative alleles observed at linked genomic locus Lᵢ,
PLᵢ0 is the probability of observing the linked reference allele at linked genomic locus Lᵢ, given allele T0, and
T0Lᵢ0, T0Lᵢ1, T1Lᵢ0 and T1Lᵢ1 are haplotype frequencies for (i) the target reference allele and the linked reference allele, (ii) the target reference allele and the linked alternative allele, (iii) the target alternative allele and the linked reference allele, and (iv) the target alternative allele and the linked alternative allele.

A12. The method of any one of embodiments A7 to A11, wherein the likelihood (L) for a homozygous target alternative allele genotype (T11) is generated according to a process represented by equation (2):

$$L ( T 11 ) = P ( D \vert T 11 ) \times P ( T 11 ) = {\prod }_{{L}_{i}} ( \begin{matrix}{L}_{i} 0 + {L}_{i} 1 \\ {L}_{i} 0\end{matrix} ) \times {PL}_{i} {0}^{{L}_{i} 0} \times {( 1 - {PL}_{i} 0 )}^{{L}_{i} 1} \times {( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} )}^{2} \qquad ( 2 )$$

wherein:
L(T11) is the likelihood of genotype 11 at target genomic locus T
Lᵢ0 is the allele quantification of linked reference alleles observed at linked genomic locus Lᵢ
Lᵢ1 is the allele quantification of linked alternative alleles observed at linked genomic locus Lᵢ
PLᵢ0 is the probability of observing the linked reference allele at linked genomic locus Lᵢ, given allele T1, and
T0Lᵢ0, T0Lᵢ1, T1Lᵢ0 and T1Lᵢ1 are haplotype frequencies for (i) the target reference allele and the linked reference allele, (ii) the target reference allele and the linked alternative allele, (iii) the target alternative allele and the linked reference allele, and (iv) the target alternative allele and the linked alternative allele.

A13. The method of embodiment A11 or A12, wherein the likelihood (L) for a heterozygous target reference allele and target alternative allele genotype (T01) is generated according to a process derived from equation (1) and equation (2).

A13.1 The method of embodiment A13, wherein the likelihood (L) for a heterozygous target reference allele and target alternative allele genotype (T01) is generated according to a process represented by equation (3):

$$L ( T 01 ) = P ( D \vert T 01 ) \times P ( T 01 ) = \prod {L}_{i} ( \begin{matrix}{L}_{i} 0 + {L}_{i} 1 \\ {L}_{i} 0\end{matrix} ) \times P {L}_{i} {0}^{{L}_{i} 0} \times {( 1 - P {L}_{i} 0 )}^{{L}_{i} 0} \times ( 2 \times ( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} ) \times ( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} ) ) \\ where : \\ {PL}_{i} 0 = ( 0.5 \times \frac{T 0 {L}_{i} 0}{T 0 {L}_{i} 0 + T 0 {L}_{i} 1} ) + ( 0.5 \times \frac{T 1 {L}_{i} 0}{T 1 {L}_{i} 0 + T 1 {L}_{i} 1} ) . \qquad ( 3 )$$

A14. The method of any one of embodiments A1 to A13.1, wherein the genotype generated in (d) is an unphased genotype.

A15. The method of any one of embodiments A1 to A14, wherein the genotype generated in (d) is for a single nucleotide polymorphism (SNP).

A16. The method of any one of embodiments A1 to A15, wherein the genotype generated in (d) is for a bi-allelic single nucleotide polymorphism (SNP).

A17. The method of any one of embodiments A1 to A16, wherein (b) comprises quantifying a plurality of linked reference alleles and quantifying a plurality of linked alternative alleles, thereby generating a plurality of allele quantifications for a plurality of linked genomic loci.

A18. The method of embodiment A17, wherein the plurality of linked genomic loci comprises loci within about 10 kilobases upstream and about 10 kilobases downstream of the target genomic locus.

A19. The method of embodiment A17 or A18, wherein the plurality of linked genomic loci comprises about 10 linked genomic loci to about 1000 linked genomic loci.

A20. The method of any one of embodiments A17 to A19, wherein a plurality of genotype likelihood sets for the target genomic locus is generated according to the plurality of allele quantifications for the plurality of linked genomic loci.

A20.1 The method of embodiment A20, wherein a composite genotype likelihood is generated for each genotype from the plurality of genotype likelihood sets.

A21. The method of embodiment A20 or A20.1, wherein the genotype at the target genomic locus is generated based on the plurality of genotype likelihood sets and/or the composite genotype likelihoods.

A22. The method of any one of embodiments A1 to A21, comprising generating a plurality of genotypes at a plurality of target genomic loci for the test sample.

A23. The method of embodiment A22, wherein the plurality of target genomic loci comprises about 100,000 loci or more.

A24. The method of embodiment A22, wherein the plurality of target genomic loci comprises about 600,000 loci or more.

A25. The method of any one of embodiments A22 to A24, wherein each genotype in the plurality of genotypes is generated independently from the other genotypes in the plurality of genotypes.

A26. The method of any one of embodiments A1 to A25, wherein generating the genotype at the target genomic locus does not comprise generating a haplotype for two or more target genomic loci.

A26.1 The method of any one of embodiments A1 to A26, wherein generating the genotype at the target genomic locus does not comprise generating a haplotype for a target genomic locus and one or more linked genomic loci.

A27. The method of any one of embodiments A22 to A26.1, further comprising identifying a subject based on the plurality of genotypes generated for the test sample.

A28. The method of any one of embodiments A1 to A27, wherein the method comprises prior to (b) filtering sequence reads.

A29. The method of embodiment A28, wherein the sequence reads are filtered by removing sequence reads that align to a reference genome locus that is within 1 to 10 bases of an insertion polymorphism or a deletion polymorphism.

A30. The method of embodiment A29, wherein the sequence reads are filtered by removing sequence reads that align to a reference genome locus that is within 4 bases of an insertion polymorphism or a deletion polymorphism.

A31. The method of any one of embodiments A22 to A27, wherein the method comprises filtering the target genomic loci.

A32. The method of embodiment A31, wherein the target genomic loci are filtered by removing genomic loci that are within 1 to 10 bases of an insertion polymorphism or a deletion polymorphism.

A33. The method of embodiment A32, wherein the target genomic loci are filtered by removing genomic loci that are within 4 bases of an insertion polymorphism or a deletion polymorphism.

A34. The method of any one of embodiments A1 to A33, further comprising prior to (a) sequencing the nucleic acid in the test sample by a sequencing process, thereby generating sequence reads.

A35. The method of embodiment A34, wherein the sequencing process is a genome-wide sequencing process.

A36. The method of embodiment A34 or A35, wherein the sequencing process is a non-targeted sequencing process.

A37. The method of any one of embodiments A34 to A36, wherein the sequencing process is a massively parallel sequencing process.

A38. The method of any one of embodiments A34 to A37, wherein the sequencing process is performed at about 2-fold coverage.

A39. The method of any one of embodiments A34 to A37, wherein the sequencing process is performed at about 1-fold coverage.

A40. The method of any one of embodiments A34 to A39, further comprising aligning the sequence reads to a reference genome, thereby generating aligned sequence reads.

A41. The method of any one of embodiments A34 to A40, further comprising prior to sequencing the nucleic acid in the test sample, producing a sequencing library.

A42. The method of embodiment A41, wherein producing a sequencing library comprises generating single-stranded nucleic acid (ssNA) from the nucleic acid in the test sample.

A43. The method of embodiment A42, wherein producing a sequencing library comprises combining the ssNA with a plurality of scaffold adapter species, or components thereof.

A44. The method of embodiment A43, wherein the scaffold adapter components comprise (i) an oligonucleotide and (ii) a scaffold polynucleotide comprising an ssNA hybridization region and an oligonucleotide hybridization region.

A45. The method of embodiment A44, wherein the ssNA and the plurality of scaffold adapter species, or components thereof, are combined under conditions in which the scaffold polynucleotide is hybridized to (i) an ssNA terminal region and (ii) the oligonucleotide, thereby forming hybridization products in which an end of the oligonucleotide is adjacent to an end of the ssNA terminal region.

A46. The method of embodiment A45, further comprising covalently linking the adjacent ends of the oligonucleotide and the ssNA terminal region, thereby generating covalently linked hybridization products.

A47. The method of any one of embodiments A44 to A46, wherein the ssNA hybridization region in the scaffold polynucleotide of each scaffold adapter species comprises a unique sequence.

A48. The method of any one of embodiments A44 to A47, wherein the ssNA hybridization region in the scaffold polynucleotide of each scaffold adapter species comprises a random sequence.

A49. The method of any one of embodiments A43 to A48, wherein one or both native ends of the ssNA are present when the ssNA is combined with the plurality of scaffold adapter species, or components thereof.

A50. The method of any one of embodiments A1 to A49, wherein the test sample is a forensic sample.

A51. The method of any one of embodiments A1 to A49, wherein the test sample is a non-forensic sample.

A52. The method embodiment A50 or A51, wherein the test sample comprises hair.

A53. The method of embodiment A50 or A51, wherein the test sample comprises bone.

A54. The method of any one of embodiments A1 to A53, wherein the test sample is from a human subject.

A55. The method of any one of embodiments A1 to A54, wherein the nucleic acid in the test sample comprises cell free nucleic acid.

A56. The method of any one of embodiments A1 to A55, wherein the nucleic acid in the test sample comprises degraded or damaged nucleic acid.

A57. The method of any one of embodiments A1 to A56, wherein the nucleic acid in the test sample comprises fragmented nucleic acid.

A58. The method of any one of embodiments A1 to A57, wherein the nucleic acid in the test sample comprises single-stranded nucleic acid, double-stranded nucleic acid, or single-stranded nucleic acid and double-stranded nucleic acid.

A59. The method of any one of embodiments A1 to A58, wherein the sequence reads are generated from single-stranded nucleic acid fragments, double-stranded nucleic acid fragments, or single-stranded nucleic acid fragments and double-stranded nucleic acid fragments, from the test sample.

A60. The method of any one of embodiments A1 to A59, wherein the one or more or all of (a), (b), (c), and (d) are performed by a computer.

B1. A method for generating a genotype for a target genomic locus for a test sample, comprising:
a) for a test sample comprising nucleic acid, obtaining sequence reads aligned to a reference genome;
b) for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group;
c) generating a haplotype pair likelihood set for the haplotype group according to i) the allele quantifications in (b), and ii) a probability of each haplotype pair; and
d) generating a genotype at the target genomic locus based on the haplotype pair likelihood set in (c).

B2. The method of embodiment 1, wherein the haplotype pair likelihood set for the haplotype group is generated in (c) according to a Bayesian probability.

B3. The method of embodiment 1 or B2, wherein the haplotype pair likelihood set for the haplotype group is generated in (c), given the allele quantifications in (b), according to i) a probability of the allele quantifications in (b) given each haplotype pair, and ii) a probability of each haplotype pair.

B4. The method of embodiment B3, wherein the probability in (i) is adjusted according to a measure of sequencing error.

B5. The method of embodiment B3 or B4, wherein the probability in (i) is determined according to which genotype is most likely observed at each genomic locus across the haplotype group, given a particular haplotype pair.

B6. The method of embodiment B5, comprising calculating the probability of the allele quantifications in (b) at each at each genomic locus and generating a product across all genomic loci in the haplotype group.

B7. The method of any one of embodiments 1 to B6, wherein the probability of each haplotype pair in (c)(ii) is determined, in part, according to haplotype frequencies.

B8. The method of embodiment B7, wherein the probability of each haplotype pair in (c)(ii) is determined, in part, according to haplotype frequencies for (i) a target reference allele and the linked reference allele, (ii) a target reference allele and the linked alternative allele, (iii) a target alternative allele and the linked reference allele, and (iv) a target alternative allele and the linked alternative allele.

B9. The method of any one of embodiments 1 to B8, wherein (b) further comprises quantifying a target reference allele and quantifying a target alternative allele, thereby generating allele quantifications for the target genomic locus.

B10. The method of any one of embodiments 1 to B9, wherein the haplotype pair likelihood set for the haplotype group is generated in (c) according to a probability that the test sample has a particular haplotype pair, i and j, given the allele quantifications in (b), D, wherein the probability, P(Hᵢ, Hⱼ|D), is derived from equation A:

$$P ( {H}_{i} , {H}_{j} \vert D ) = \frac{P ( D \vert {H}_{i} , {H}_{j} ) \times P ( {H}_{i} , {H}_{j} )}{P ( D )} \qquad ( A )$$

wherein P(D|Hᵢ, Hⱼ) is the probability of the allele quantifications in (b), given the allele quantifications derive from haplotype pair Hᵢ, Hⱼ; P(Hᵢ, Hⱼ) is the probability of each haplotype pair derived from haplotype frequencies; and P(D) is the probability of the allele quantifications in (b).

B11. The method of embodiment 10, wherein P(D|Hᵢ, Hⱼ) is determined according to which genotype is most likely observed at each genomic locus, s, across the haplotype group, given haplotype pair Hᵢ, Hⱼ is present.

B12. The method of embodiment B11, comprising calculating the probability of the allele quantifications in (b), D, at each at each genomic locus, s, and generating a product across all genomic loci in the haplotype group according to equation B:

$$P ( D \vert {H}_{i} , {H}_{j} ) = {\prod }_{s = 1}^{n} P ( {D}_{s} \vert {H}_{i s} , {H}_{j s} ) . \qquad ( B )$$

B13. The method of any one of embodiments 1 to B12, wherein (c) further comprises identifying the most probable haplotype pair from the haplotype pair likelihood set.

B14. The method of embodiment B13, wherein the genotype at the target genomic locus is generated in (d) according to the most probable haplotype pair.

B15. The method of any one of embodiments 1 to B12, wherein (c) further comprises aggregating the haplotype pair likelihoods across all haplotype pairs for the haplotype group, thereby generating aggregate likelihoods.

B16. The method embodiment B15, wherein the genotype at the target genomic locus is generated in (d) according to the highest aggregate likelihood.

B17. The method of any one of embodiments 1 to B16, wherein the genotype at the target genomic locus is chosen from homozygous for a target reference allele, heterozygous for a target reference allele and a target alternative allele, and homozygous for a target alternative allele.

B18. The method of any one of embodiments 1 to B17, wherein the genotype generated in (d) is for a single nucleotide polymorphism (SNP).

B19. The method of any one of embodiments 1 to B18, wherein the genotype generated in (d) is for a bi-allelic single nucleotide polymorphism (SNP).

B20. The method of any one of embodiments 1 to B19, wherein (b) comprises, for a plurality of haplotype groups each comprising a target genomic locus and a plurality of linked genomic loci, quantifying a linked reference allele and quantifying a linked alternative allele for each linked genomic locus in each group according to the sequence reads generated in (a), thereby generating allele quantifications for each linked genomic locus for each group in the plurality of haplotype groups.

B21. The method of embodiment B20, wherein a plurality of haplotype pair likelihood sets are generated in (c) according to the allele quantifications for each linked genomic locus for each group in the plurality of haplotype groups.

B22. The method of embodiment B21, wherein a plurality of genotypes at a plurality of target genomic loci are generated in (d) based on the plurality of haplotype pair likelihood sets.

B23. The method of embodiment B22, wherein the plurality of target genomic loci comprises about 100,000 loci or more.

B24. The method of embodiment B22, wherein the plurality of target genomic loci comprises about 600,000 loci or more.

B25. The method of any one of embodiments B22 to B24, wherein each genotype in the plurality of genotypes is generated independently from the other genotypes in the plurality of genotypes.

B26. The method of any one of embodiments B22 to B25, further comprising identifying a subject based on the plurality of genotypes generated for the test sample.

B27. The method of any one of embodiments B22 to B26, wherein the method comprises filtering the target genomic loci.

B28. The method of embodiment B27, wherein the target genomic loci are filtered by removing genomic loci that are within 1 to 10 bases of an insertion polymorphism or a deletion polymorphism.

B29. The method of embodiment B28, wherein the target genomic loci are filtered by removing genomic loci that are within 4 bases of an insertion polymorphism or a deletion polymorphism.

B30. The method of any one of embodiments 1 to B29, wherein generating the genotype at the target genomic locus does not comprise generating a haplotype group comprising two or more target genomic loci.

B31. The method of any one of embodiments 1 to B30, wherein the plurality of linked genomic loci in the haplotype group comprises loci within about 10 kilobases upstream and about 10 kilobases downstream of the target genomic locus.

B32. The method of any one of embodiments 1 to B31, wherein the plurality of linked genomic loci in the haplotype group comprises about 10 linked genomic loci to about 1000 linked genomic loci.

B33. The method of any one of embodiments 1 to B31, wherein the plurality of linked genomic loci in the haplotype group comprises about 5 linked genomic loci to about 50 linked genomic loci.

B34. The method of any one of embodiments 1 to B33, wherein each locus in the plurality of linked genomic loci in the haplotype group is at least about 70 bases away from other loci in the haplotype group.

B35. The method of any one of embodiments 1 to B34, wherein the method comprises prior to (b) filtering sequence reads.

B36. The method of embodiment B35, wherein the sequence reads are filtered by removing sequence reads that align to a reference genome locus that is within 1 to 10 bases of an insertion polymorphism or a deletion polymorphism.

B37. The method of embodiment B36, wherein the sequence reads are filtered by removing sequence reads that align to a reference genome locus that is within 4 bases of an insertion polymorphism or a deletion polymorphism.

B38. The method of any one of embodiments 1 to B37, further comprising prior to (a) sequencing the nucleic acid in the test sample by a sequencing process, thereby generating sequence reads.

B39. The method of embodiment B38, wherein the sequencing process is a genome-wide sequencing process.

B40. The method of embodiment B38 or B39, wherein the sequencing process is a non-targeted sequencing process.

B41. The method of any one of embodiments B38 to B40, wherein the sequencing process is a massively parallel sequencing process.

B42. The method of any one of embodiments B38 to B41, wherein the sequencing process is performed at about 2-fold coverage.

B43. The method of any one of embodiments B38 to B41, wherein the sequencing process is performed at about 1-fold coverage.

B44. The method of any one of embodiments B38 to B43, further comprising aligning the sequence reads to a reference genome, thereby generating aligned sequence reads.

B45. The method of any one of embodiments B38 to B44, further comprising prior to sequencing the nucleic acid in the test sample, producing a sequencing library.

B46. The method of embodiment B45, wherein producing a sequencing library comprises generating single-stranded nucleic acid (ssNA) from the nucleic acid in the test sample.

B47. The method of embodiment B46, wherein producing a sequencing library comprises combining the ssNA with a plurality of scaffold adapter species, or components thereof.

B48. The method of embodiment B47, wherein the scaffold adapter components comprise (i) an oligonucleotide and (ii) a scaffold polynucleotide comprising an ssNA hybridization region and an oligonucleotide hybridization region.

B49. The method of embodiment B48, wherein the ssNA and the plurality of scaffold adapter species, or components thereof, are combined under conditions in which the scaffold polynucleotide is hybridized to (i) an ssNA terminal region and (ii) the oligonucleotide, thereby forming hybridization products in which an end of the oligonucleotide is adjacent to an end of the ssNA terminal region.

B50. The method of embodiment B49, further comprising covalently linking the adjacent ends of the oligonucleotide and the ssNA terminal region, thereby generating covalently linked hybridization products.

B51. The method of any one of embodiments B48 to B50, wherein the ssNA hybridization region in the scaffold polynucleotide of each scaffold adapter species comprises a unique sequence.

B52. The method of any one of embodiments B48 to B51, wherein the ssNA hybridization region in the scaffold polynucleotide of each scaffold adapter species comprises a random sequence.

B53. The method of any one of embodiments B47 to B52, wherein one or both native ends of the ssNA are present when the ssNA is combined with the plurality of scaffold adapter species, or components thereof.

B54. The method of any one of embodiments 1 to B53, wherein the test sample is a forensic sample.

B55. The method of any one of embodiments 1 to B53, wherein the test sample is a non-forensic sample.

B56. The method embodiment B54 or B55, wherein the test sample comprises hair.

B57. The method of embodiment B54 or B55, wherein the test sample comprises bone.

B58. The method of any one of embodiments 1 to B57, wherein the test sample is from a human subject.

B59. The method of any one of embodiments 1 to B58, wherein the nucleic acid in the test sample comprises cell free nucleic acid.

B60. The method of any one of embodiments 1 to B59, wherein the nucleic acid in the test sample comprises degraded or damaged nucleic acid.

B61. The method of any one of embodiments 1 to B60, wherein the nucleic acid in the test sample comprises fragmented nucleic acid.

B62. The method of any one of embodiments 1 to B61, wherein the nucleic acid in the test sample comprises single-stranded nucleic acid, double-stranded nucleic acid, or single-stranded nucleic acid and double-stranded nucleic acid.

B63. The method of any one of embodiments 1 to B62, wherein the sequence reads are generated from single-stranded nucleic acid fragments, double-stranded nucleic acid fragments, or single-stranded nucleic acid fragments and double-stranded nucleic acid fragments, from the test sample.

B64. The method of any one of embodiments 1 to B63, wherein the one or more or all of (a), (b), (c), and (d) are performed by a computer.

### EXAMPLES

The examples set forth below illustrate certain implementations and do not limit the technology.

### Example 1: A Fast Procedure for Genotype Inference from Low Coverage and Fragmented DNA Sequence Data and Haplotype Panels

Databases of genotype information for millions of individuals are available for search and analysis. These databases include GEDMATCH and FAMILYTREEDNA. The genotype data in these databases often is contributed by users of direct to consumer (DTC) genetic testing companies such as, for example, 23ANDME or ANCESTRYDNA. The original concept for the more open databases was to provide a platform for genealogy enthusiasts to do DNA based relative finding across platforms. For example, such databases allow a user who may have ANCESTRYDNA genotype data to find a cousin who may have 23ANDME genotype data.

Recently, law enforcement have recognized the potential of genetic genealogy to assist with solving crime. It is often possible to perform a genotype analysis on a forensic sample like blood or semen. Typically, the same genotype array technology used by direct to consumer companies is used to genotype forensic samples. However, many forensic samples (e.g., hair, bone) do not contain enough good quality DNA for use on a genotype array.

This Example describes a procedure that produces accurate genotypes from very low coverage shotgun sequence data. Furthermore, this approach works even when the DNA sequence data is derived from highly fragmented and chemically damaged DNA molecules, as is often found in forensic samples. In certain instances, sequencing libraries are generated from highly fragmented and chemically damaged DNA molecules using scaffold adapters described herein.

The approach to infer genotypes at a defined set of target site positions is outlined below:

1. Generate DNA sequence reads using low coverage shotgun sequencing. Typically, about 2 fold coverage of the genome is used as input data. This fold coverage input level may decrease as imputation panels grow. A 2 fold coverage of the genome means that each position in the genome is observed, on average, 2 times. However, as each DNA sequence read is from a random position on the genome, some genomic positions are not observed in any DNA sequence read. Some genomic positions are observed once or only a few times.

2. Align the DNA sequence reads to a reference genome to determine which allelic observations are present in the data at known polymorphic positions. Known polymorphic positions are available from large human genome sequencing projects (e.g., the 1000 Genomes project).

3. Filter the allelic observations to remove sites that are likely in error because they are nearby a known, high-frequency insertion or deletion polymorphism. Mis-alignment between DNA sequence reads and a reference genome can lead to mis-calling of allelic observations. This problem is exacerbated when there is an insertion or deletion difference between the reference genome and the DNA sequence that is being aligned. To mitigate this problem, regions of known, high-frequency insertion or deletion polymorphisms are removed from the list of observations as they may be called incorrectly.

4. Use haplotype frequencies between each target site and each linked site observed from the input panel (1000 Genomes, for example) to determine the likelihood of the observed alleles under each possible target site genotype. The degree of information from each linked site is related to the linkage disequilibrium between it and the target site, and to how many allelic observations were made at the linked site. Additionally, allelic observations at the target site itself can be handled in this framework by considering the target site as perfectly linked to itself. The algorithmic details of this procedure are as follows:
a) Generate a list of genomic sites/markers for the assay. This list of genomic sites may be referred to as a target list. A target list typically derives from markers present on ANCESTRYDNA, 23ANDME, FAMILTYTREE DNA or other direct-to-consumer array platforms.
b) Generate a table of linked sites (L=L1, L2, . . . Ln) for each target site. These are sites nearby one or more target sites (see FIG. 1). In this Example, the window for linked sites is 10 kb upstream and 10 kb downstream for each target site. The number of linked sites for each target site varies (e.g., tens to hundreds of linked sites per target site). The list of linked sites may be generated based on any large panel of human genome variation (e.g., the 1000 Genomes Project data as used in this Example).

The table of target sites and linked sites include haplotype frequencies of each linked site allele and the target site allele(s) to which it is linked. The format of this table in the current implementation is a tab-delimited file with the following columns:
1. Chr of target site
2. position of target site
3. ID of target site (usually an rsID)
4. Chr of linked site
5. Position of linked site
6. ID of linked site (usually an rsID)
7. Reference allele at linked site (expressed as a particular nucleotide base)
8. Alternative allele at linked site (expressed as a particular nucleotide base)
9. T0L0 frequency (haplotype frequency of target=ref, linked=ref)
10. T0L1 frequency (haplotype frequency of target=ref, linked=alt)
11. T1L0 frequency (haplotype frequency of target=alt, linked=ref)
12. T1 L1 frequency (haplotype frequency of target=alt, linked=alt)

The above table essentially is a fixed table that is input to a genotype inference algorithm. That is, it generally need only be generated once.

In the above table, 0 refers to the reference allele and 1 refers to the alternative allele. Haplotype frequencies can be used to measure the amount of linkage disequilibrium and generally refer to counts of pairs of alleles (i.e., haplotypes) in a population. Haplotype frequencies may be referred to a haplotype counts. The haplotype frequencies in columns 9-12 are obtained from a database (e.g., any database containing haplotype frequency data). In this Example, the haplotype frequencies in columns 9-12 are obtained from the 1000 Genomes Project public database.

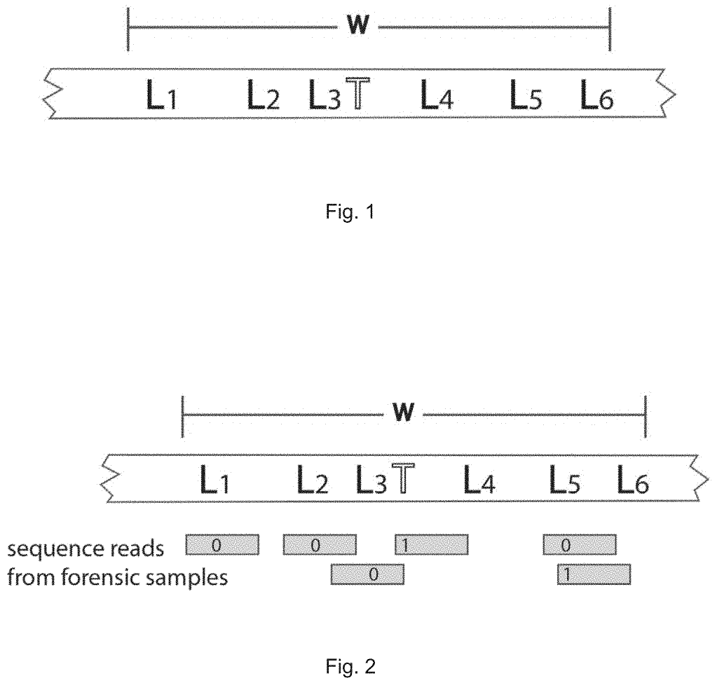

A target site is considered a site that is linked to itself. In this implementation, there are only T0L0 and T1L1 haplotype counts, and columns 10 and 11 necessarily have value 0. In this way, each target site is perfectly linked to itself. As described below, this allows use of the allelic observations at target sites in the same mathematical framework as allelic observations at linked sites. In certain implementations, a blacklist is used to remove target and linked sites. A blacklist of regions may be constructed around known insertion or deletion polymorphisms. These sites sometimes generate incorrect alignments and lead to incorrect allele calls at target or linked sites.
c) Map sequence data to a reference genome to generate allelic observations at target sites and linked sites (see FIG. 2). In this Example, the BWA-MEM aligner is used, which can allow multiple mismatches, depending on the length of the sequence being aligned. In certain implementations, sequences are filtered to exclude those whose alignment score is below a certain threshold. In certain implementations, the quality score of each base is used to exclude those that are more likely to be errors, and certain implementations incorporate a fixed background of base-calling error. The allelic observations at target sites and linked sites are added to the haplotype table generating the following new columns at each Linked site line.
13. Counts of L0 observations, i.e., reads carrying the reference allele
14. Counts of L1 observations, i.e., reads carrying the alternative allele

The counts in columns 13 and 14 are observed counts of alleles in a test sample (e.g., a forensic sample).
d) At this point, all of the required information is now in the table. Use the information (i.e., columns 13 and 14) from the allelic observations at the target site and each linked site along with the linkage information (i.e., columns 9-12) from the panel to compute the likelihood of each possible target site genotype. There are three possible unphased genotypes (phased genotype data is not needed for this genotyping method) for each bi-allelic target site:
T00=homozygous for the reference allele
T01=heterozygous
T11=homozygous for the alternative allele

The likelihood for each unphased genotype at each target site is computed using a Bayesian approach. The probability of the data under each target site genotype model is determined. Then, that probability is multiplied by the prior probability of each genotype using the target site genotype frequencies and the Hardy-Weinberg assumption of genotype frequencies given allele frequencies. The Hardy-Weinberg assumption is given allele frequencies of p and q=1−p for a bi-allelic site, the probability of each possible genotype is: homozygous for p=p*p; heterozygous for p, q=2*p*q; homozygous for q=q*q. These values are used as prior probabilities in the Bayesian formulas below (i.e., the term in parenthesis that is squared; the bit in the parenthesis is a way of measuring p for the reference allele). The entire term in parenthesis yields a particular allele frequency of the target site reference allele (in equation (1)) or target site alternative allele (in equation (2)). For example, in equation (1) below, T0Lᵢ0+T0Lᵢ1 is divided by all four haplotype frequencies (T0Lᵢ0, T0Lᵢ1, T1Lᵢ0 and T1Lᵢ1). In other words, the number of haplotypes that carry T0—the reference allele at the target site—is divided by all the haplotypes. That provides the frequency of the reference allele at the target site.

The likelihood L of genotype 00 at target site T is calculated as follows:

$$AB L ( T 00 ) = P ( D \vert T 00 ) \times P ( T 00 ) = {\prod }_{{L}_{i}} ( \begin{matrix}{L}_{i} 0 + {L}_{i} 1 \\ {L}_{i} 0\end{matrix} ) \times {PL}_{i} {0}^{{L}_{i} 0} \times {( 1 - P {L}_{i} 0 )}^{{L}_{i} 1} \times {( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} )}^{2} CDEF \qquad ( 1 )$$

Where:
Operation A is the probability of the observed data (D) given the genotype at target site T is 00.
Operation B is the probability that the genotype at the target site T is 00 (also referred to as the “prior” for genotype 00 at the target site).
The product of operations C, D, and E are equal to operation A. Operations C, D, and E are a way to calculate operation A given the observations at the linked sites and the haplotype panel data. More specifically, operations C, D, and E are the binomial sampling probabilities for the observations at each linked site Lᵢ given the haplotype information. The product term refers to each site evaluated independently and then all sites are multiplied together.
Operation F is the term used to calculate operation B. Given the haplotype counts in the table, this is a way to learn the frequency of homozygous reference at the target site. The term in parenthesis calculates the frequency of the 0 allele at site T. Taking the square of that gives the frequency of homozygotes in the population, under the Hardy-Weinberg assumption.
L(T00) is the likelihood of genotype 00 at target site T.
D refers to the data, i.e., the observed alleles in the last two columns (columns 13 and 14).
Lᵢ0 is the count of reference alleles observed at linked site Lᵢ.
Lᵢ1 is the count of alternative alleles observed at linked site Lᵢ.
PLᵢ0 is the probability of observing the reference allele at linked site Lᵢ, given allele T0. The probability is determined from haplotype frequencies. This probability can be adjusted to account for sequencing error (e.g., using a fixed background rate of sequencing error).
T0Lᵢ0, T0Lᵢ1, T1Lᵢ0 and T1Lᵢ1 are the haplotype frequencies listed under columns 9-12, respectively.

The likelihoods for the two other genotypes, T01 and T11, are calculated similarly. T01 is calculated under the model that observed alleles are equally likely to derive from either chromosome.

The likelihood L of genotype 11 at target site T is calculated as follows:

$$G H L ( T 11 ) = P ( D \vert T 11 ) \times P ( T 11 ) = {\prod }_{{L}_{i}} ( \begin{matrix}{L}_{i} 0 + {L}_{i} 1 \\ {L}_{i} 0\end{matrix} ) \times {PL}_{i} {0}^{{L}_{i} 0} \times {( 1 - P {L}_{i} 0 )}^{{L}_{i} 1} \times {( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} )}^{2} \\ IJKL \qquad ( 2 )$$

Where:
Operation G is the probability of the observed data (D) given the genotype at target site T is 11.
Operation H is the probability that the genotype at the target site T is 11 (also referred to as the “prior” for genotype 11 at the target site).
The product of operations I, J, and K are equal to operation G. Operations I, J, and K are a way to calculate operation G given the observations at the linked sites and the haplotype panel data. More specifically, operations I, J, and K are the binomial sampling probabilities for the observations at each linked site Lᵢ given the haplotype information. The product term refers to each site evaluated independently and then all sites are multiplied together.
Operation L is the term used to calculate operation H. Given the haplotype counts in the table, this is a way to learn the frequency of homozygous alternative at the target site. The term in parenthesis calculates the frequency of the 1 allele at site T. Taking the square of that gives the frequency of homozygotes in the population, under the Hardy-Weinberg assumption.
L(T11) is the likelihood of genotype 11 at target site T.
D refers to the data, i.e., the observed alleles in the last two columns (columns 13 and 14).
Lᵢ0 is the count of reference alleles observed at linked site Lᵢ.
Lᵢ1 is the count of alternative alleles observed at linked site Lᵢ.
PLᵢ0 is the probability of observing the reference allele at linked site Lᵢ, given allele T1. The probability is determined from haplotype frequencies. This probability can be adjusted to account for sequencing error (e.g., using a fixed background rate of sequencing error).
T0Lᵢ0, T0Lᵢ1, T1Lᵢ0 and Tᵢ1 are the haplotype frequencies listed under columns 9-12, respectively.

Finally, the likelihood L that the target site T is heterozygous (01) for the reference and alternative alleles is calculated according to a process derived from equation (1) and equation (2), and by assuming each observed allele has probability of 0.5 of coming from either parental genome copy.

Thus:

$$L ( T 01 ) = P ( D \vert T 01 ) \times P ( T 01 ) = \prod {L}_{i} ( \begin{matrix}{L}_{i} 0 + {L}_{i} 1 \\ {L}_{i} 0\end{matrix} ) \times P {L}_{i} {0}^{{L}_{i} 0} \times {( 1 - P {L}_{i} 0 )}^{{L}_{i} 0} \times ( 2 \times ( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} ) \times ( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} ) ) \\ where : \\ {PL}_{i} 0 = ( 0.5 \times \frac{T 0 {L}_{i} 0}{T 0 {L}_{i} 0 + T 0 {L}_{i} 1} ) + ( 0.5 \times \frac{T 1 {L}_{i} 0}{T 1 {L}_{i} 0 + T 1 {L}_{i} 1} ) \qquad ( 3 )$$

In equation (3), the PLᵢ0 terms are calculated by assuming each observation at the linked site has equal probability of coming from either of the two haplotypes since the target site is heterozygous (half the DNA must be maternally derived and half must be paternally derived). The term at the end of equation 3 (starting with (2× . . . )) is the Hardy-Weinberg term for probability of being a heterozygote given an allele frequency (i.e., 2*p*q).
e) Determine the genotype at each target site by choosing the most likely genotype as calculated above. The likelihood of the most likely genotype is compared to the likelihood of the second most likely genotype to generate a likelihood ratio. Genotype calls can be filtered on this ratio, calling only genotypes with an arbitrarily high likelihood ratio. When this likelihood ratio is below the cutoff, it is still possible to call one of the alleles to output a partial genotype.

5. Because each DNA sequence read is independent data from other sequence reads, genotype likelihoods calculated from data at each linked site are treated as independent observations. A composite likelihood is generated by multiplying all linked site likelihoods for each target site genotype. Optionally, one can filter for a level of linkage disequilibrium and observed coverage. Comparison of the composite likelihoods of each target site genotype (homozygous reference, heterozygous, and homozygous alternative) yields a genotype likelihood ratio. This number can be used to filter results at any desired confidence level.

6. Convert genotypes into a file format suitable for downstream analysis (e.g., upload to an open genetic genealogy service).

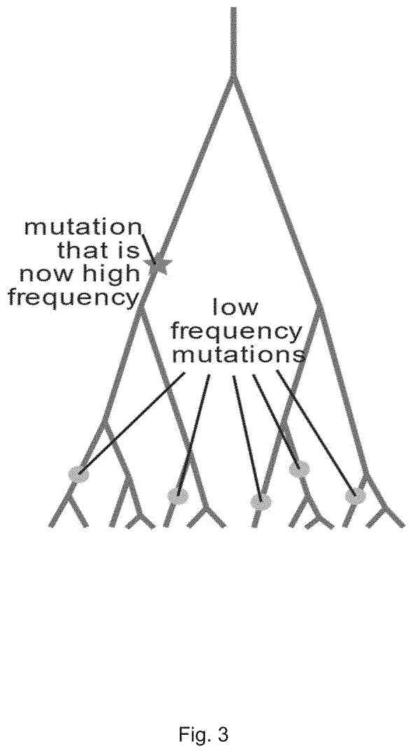

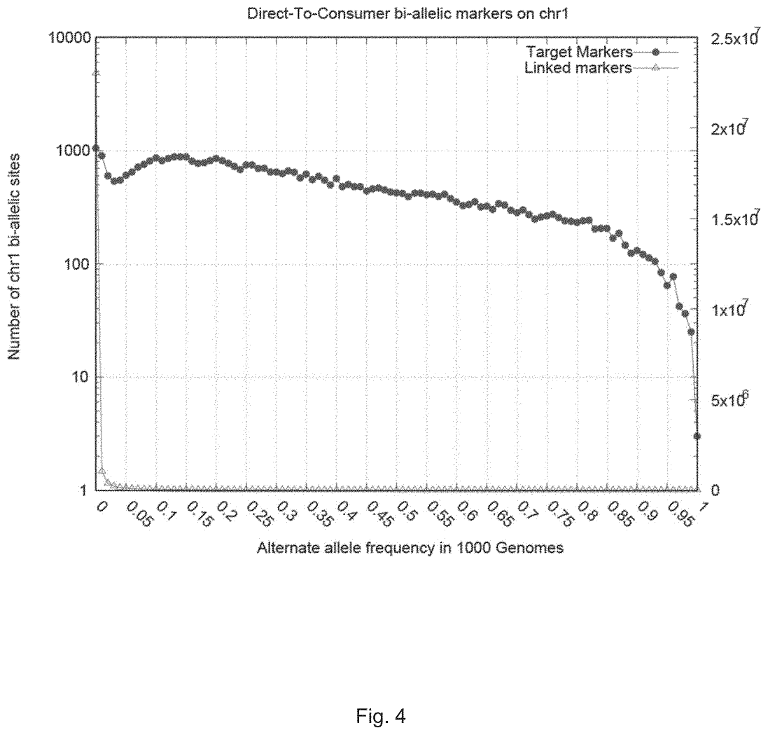

This framework of using low-coverage sequence data (about 2-fold genome coverage) at target sites and linked sites works well for certain conditions of interest. If the target site is a position of high minor-allele frequency, it is expected that the mutation generating the minor allele is old. Therefore, the mutation sits higher in the phylogenetic tree than a mutation of lower population frequency. Random genome data generates information at many sites of lower allele frequency due to the distribution of allele frequencies in humans, i.e., most genetic variation is rare. There is generally more information from rare alleles about nearby high-frequency alleles than the other way around. This is illustrated in FIG. 3, which shows haplotypes at any genomic region exist within the context of a phylogenetic tree, although the topology of the tree may not be known. Target sites (e.g., from direct to consumer arrays) typically have high allele frequencies and are thus older in time and deeper in the tree. Most genetic variation exists as lower frequency alleles and thus falls toward the bottom of the tree. As shown in the FIG. 3, it is often possible to determine which upper branch of the tree a haplotype sits on given some information about lower branches. The opposite is not true. Thus, target site genotypes often can be ascertained from one or more low-frequency linked sites, but generally not the opposite. Since genotypes are determined at target sites that were selected for direct to consumer arrays, i.e., mostly high minor-allele-frequency sites, using data from large panels of more rare alleles can generate accurate genotypes.

Results

Nucleic acid from a forensic test sample was converted to single-stranded DNA and a sequencing library was generated using scaffold adapters described herein. Briefly, nucleic acid extracted from a forensic test sample was incubated with a solution containing SSB at 95° C. for 5 minutes and shock cooled on ice for 2 minutes. Scaffold adapters compatible with Illumina-brand sequencers were added directly to the nucleic acid-SSB mixture, followed by the addition of a master mix containing T4 polynucleotide kinase, T4 DNA ligase, T4 DNA ligase buffer and PEG 8000. The mixture was incubated at 37° C. for 1 hour. The ligation product was purified using SPRI breads to remove excess adapters and short unligated product. The purified ligation product was amplified and multiplexing barcodes referred to as indexes were added in a PCR reaction. The PCR product was purified using 1.2×18% SPRI beads. The purified PCR product represented the completed sequencing library, which was sequenced on an Illumina sequencing platform.

A genotype calling strategy was implemented using the genotyping method described above. Specifically, the genotyping strategy described in this Example was used to determine genotypes at sites on a direct-to-consumer platform using low-coverage (i.e., about 2 fold) shotgun sequencing data. Comparison to the genotype array data as downloaded from the direct-to-consumer platform (from the same sample source) was done to determine the overall level of genotype concordance. Analysis was restricted to bi-allelic sites. Target sites less than 4 base-pairs from a known insertion or deletion variant were removed due to difficulties with alignment with low-coverage data around insertions or deletions. Typically, direct to consumer arrays genotype about 700,000 target sites. The approach described in this Example, using suitable cutoffs, calls about 92% of the target sites that are bi-allelic SNPs. Typically, all or most of the target site genotype calls generated by the approach described in this Example are needed for a positive identification of a subject from low coverage sequencing data generated for nucleic acid in a test sample (e.g., a forensic sample originating from the subject).

Genotyping method calls (i.e., results generated by the genotyping method described in this Example) were compared to results generated from an existing genotyping method (i.e., IMPUTE2), and the results are shown in Table 1 below. The comparison showed the genotyping approach described in this Example has a higher concordance with genotype array data from the same donor vs. the existing genotyping approach.

TABLE 1

| Genotype | Imputation | Genotyping | IMPUTE2-based |
|---|---|---|---|
| array | software | method | method |
| genotype | genotype | calls | calls |
| Homozygous | Homozygous | 349,113 | 340,748 |
| site | (correct allele) | | |
| | Homozygous | 1,577 | 2,604 |
| | (incorrect allele) | | |
| | Heterozygous | 16,923 | 15,513 |
| | 1 allele called | 51,809 | 100,397 |
| | (correct) | | |
| | 1 allele called | 1,571 | 1,111 |
| | (incorrect) | | |
| Heterozygous | Heterozygous | 155,188 | 153,888 |
| site | Homozygous | 7,343 | 3,542 |
| | 1 allele called | 27,347 | 29,937 |
| | (correctly) | | |
| | 1 allele called | 0 | 47 |
| | (incorrectly) | | |

### Example 2: An Alternative Approach for Genotype Calling from Low-Coverage Sequence Data

The method described in Example 1 uses information from sites that are in linkage disequilibrium with a target site to improve genotype calling. That method considers observed data at each linked site as an independent measure of alleles present at a target site. An alternative approach described in this Example uses haplotypes—sections of linked alleles—around a target site. Large panels of haplotypes are available from 1000 Genomes Project and other sources. In this approach, instead of considering the data at each linked site separately, haplotypes are considered in aggregate to generate target site genotype calls.

Below is an example workflow for this method.

1. Around each target site, haplotype sets are generated using observed haplotypes from an external reference panel such as 1000 Genomes. Each target site is described by a set of haplotypes. The sites used to describe haplotypes can be:
a) selected as commonly present in DNA recovered from certain types of samples (e.g., hair);
b) selected for high linkage disequilibrium with the target site;
c) selected for suitable mapping characteristics (e.g., avoiding repetitive regions, insertion-deletion polymorphisms, and other genome features that disrupt accurate mapping); and/or
d) spaced such that a single read does not give information about multiple sites thus avoiding over counting information from single reads.

2. DNA sequence reads from a sample (e.g., hair) are mapped to the reference human genome.

3. For each target site, it is assumed the sample is contributed from a single, diploid individual. Thus, the haplotype pair that is most probable is found given the observed bases at each site, for each haplotype around each target.

The probability of a pair of haplotypes is calculated using a Bayesian approach. The probability of each haplotype pair, given the data is the probability of the data, given each haplotype pair times the probability of the haplotype pair. The probability of the haplotype pair can be learned from the haplotype frequencies in the reference panel.

Thus, given a set of observed haplotypes:

Hᵢ=[0|1,0|1, . . . ]

Where alleles are encoded by 0=reference allele and 1=alternative allele and Hᵢₛ is the s^{th} allele on the i^{th} haplotype,

The probability that the sample derives from any particular pair of haplotypes, i and j, given observed data, D, can be derived:

$$P ( {H}_{i} , {H}_{j} \vert D ) = \frac{P ( D \vert {H}_{i} , {H}_{j} ) \times P ( {H}_{i} , {H}_{j} )}{P ( D )} \qquad ( A )$$

P(Hᵢ, Hⱼ) is observed from the reference panel. That is, it is the probability of this particular haplotype pair in the panel, independent of any data.

P(D|Hᵢ, Hⱼ) is the probability of the observed data, given that it derives from this particular haplotype pair. This term can be calculated by considering what genotype should be observed at each site, s, across the region, given that these two haplotypes are present. The probability of the data, D, is calculated at each site and the product is taken across all sites, thusly:

$$P ( D \vert {H}_{i} , {H}_{j} ) = {\prod }_{s = 1}^{n} P ( {D}_{s} \vert {H}_{i s} , {H}_{j s} ) \qquad ( B )$$

Below is an additional example workflow for this method.

Given a set of observed haplotypes, as shown below:

| | | | | |
|---|---|---|---|---|
| ## TARGET | 60523 | T | G | rs112920234 |
| 10 | 60523 | T | G | rs112920234 |
| 10 | 60753 | C | T | rs554788161 |
| 10 | 60803 | T | G | rs536478188 |
| 10 | 60969 | C | A | rs61838556 |
| 10 | 61020 | G | C | rs115033199 |
| 10 | 61331 | A | G | rs548639866 |
| 10 | 61386 | G | A | rs536439816 |
| 10 | 61614 | G | C | rs554243250 |
| 10 | 61694 | T | G | rs546443136 |
| 10 | 61836 | G | T | rs563066628 |
| 10 | 62010 | C | T | rs568474105 |
| 10 | 62068 | A | C | rs538488924 |
| 10 | 62450 | G | A | rs539912981 |
| 10 | 62554 | T | C | rs577354278 |

## Haplotypes

| | |
|---|---|
| 2394 | 00010000000000 |
| 2346 | 00000000000000 |
| 72 | 10000000001000 |
| 63 | 00000100000000 |
| 49 | 00100010100000 |
| 25 | 00011000000000 |
| 18 | 10000000000000 |
| 14 | 00000001000000 |
| 8 | 00000000000100 |
| 4 | 00010000000010 |
| 3 | 00010000000100 |
| 3 | 00000000000001 |
| 2 | 00000000010000 |
| 2 | 00010010100000 |
| 1 | 11000000001000 |
| 1 | 00001000000000 |
| 1 | 01000000000000 |

where the columns in the haplotype matrix are the sites, as listed in the order above the haplotypes and the genotype of a site at a haplotype is encoded as 0=reference and 1=alternative allele.

For each target site (genomic position for which a genotype is deduced), a haplotype description is generated that describes the linked genomic variants in a population. A haplotype description can be from 1000 Genomes or any large panel of known genome sequences. The sites that go into the haplotype description can be chosen such that they are in linkage disequilibrium with the target site and are generally recoverable from a particular type of test sample (e.g., hair DNA). The haplotypes can be described as a matrix where the rows are unique haplotypes and the columns are the genomic positions in the haplotypes. Each matrix element describes the allele of a particular site on a particular haplotype (see Table 2 below, where 0 refers to a reference allele and 1 refers to an alternative allele).

TABLE 2

| | Genomic | Genomic | Genomic | Genomic | Genomic |
|---|---|---|---|---|---|
| | position | position | position | position | position |
| | i | ii | iii | iv | v |
| Haplotype A | 0 | 0 | 0 | 0 | 0 |
| Haplotype B | 1 | 0 | 0 | 0 | 0 |
| Haplotype C | 1 | 1 | 0 | 0 | 0 |
| Haplotype D | 1 | 1 | 1 | 0 | 0 |
| Haplotype E | 1 | 1 | 1 | 1 | 0 |
| Haplotype F | 1 | 1 | 1 | 1 | 1 |
| Etc. | | | | | |

For a given sample (e.g., from a hair) reads are aligned to a reference genome and collect allelic observations at linked sites in a haplotype around a target region.

For a given sample, for each pair of haplotypes in the haplotype matrix, the probability of the observed sequence data given that they derive from that pair of haplotypes is calculated. This can be done for all pairs of haplotypes in the matrix in an all versus all comparison; the pairs where both haplotypes are the same are also included in the comparison. For example, in Table 2, a haplotype pair may be any two haplotypes from A-F (e.g., haplotype pair AB, BC, AC, . . . or AA, BB, CC, . . . ). Humans are diploid species that have two copies of each chromosome region, i.e., haplotype. Thus, the data is derived from two haplotypes. One component of this method is to determine what the two haplotypes were.

For any combination of haplotypes, it can be deduced what the target genotype is. The target site genotype can be determined by finding the haplotype pair that most probably explains the observed sample data. Once that haplotype pair is found, the target site genotype is determined by taking the target site genotype from each of the haplotypes in this haplotype pair. For example, in Table 2, if the target site is at genomic position iv, and the most likely haplotype pair given the data is haplotype pair AE, then the genotype at the target site is 0,1.

A useful variation of this procedure is instead of finding a most probable pair of haplotypes for explaining the observed data, the probability across all haplotype pairs can be aggregated. Each haplotype pair corresponds to one of three possible target genotypes (homozygous reference, heterozygous, and homozygous alternative). The probability of the data given a haplotype pair, once calculated as above, is added to the aggregate probability of the corresponding target site genotype. The target site genotype is then selected as the one with the highest aggregate probability. In this procedure, instead of finding the “best two” haplotypes for explaining the data, a comprehensive census is taken across all haplotype combinations.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%).

Certain implementations of the technology are set forth in the claim(s) that follow(s).

1. A method for generating a genotype for a target genomic locus for a test sample, comprising:
a) for a test sample comprising nucleic acid, obtaining sequence reads aligned to a reference genome;
b) from the sequence reads, quantifying a linked reference allele and quantifying a linked alternative allele, thereby generating allele quantifications for a linked genomic locus;
c) generating a set of genotype likelihoods for a target reference allele and a target alternative allele at the target genomic locus according to 1) a probability of the allele quantifications in (b), given a particular genotype at the target genomic locus, and 2) a probability of a genotype at the target genomic locus based on prior probabilities of the target reference allele and the target alternative allele; and
d) generating a genotype at the target genomic locus based on the set of genotype likelihoods.

2. The method of claim 1, wherein the probability in (c)(1) is generated according to (i) a probability of observing the linked reference allele at the linked genomic locus, given a target reference allele at the target genomic locus, and/or (ii) a probability of observing the linked reference allele at the linked genomic locus, given a target alternative allele at the target genomic locus.

3. The method of claim 2, wherein the probability in (i) is based, in part, on a measure of linkage disequilibrium for the linked reference allele and the target reference allele, and the probability in (ii) is based, in part, on a measure of linkage disequilibrium for the linked reference allele and the target alternative allele, wherein the measure of disequilibrium is based on a haplotype frequency.

4. (canceled)

5. (canceled)

6. The method of claim 1, wherein the probability in (c)(2) is based, in part, on haplotype frequencies for (i) the target reference allele and the linked reference allele, (ii) the target reference allele and the linked alternative allele, (iii) the target alternative allele and the linked reference allele, and (iv) the target alternative allele and the linked alternative allele.

7. The method of claim 1, wherein (b) comprises quantifying a plurality of linked reference alleles and quantifying a plurality of linked alternative alleles, thereby generating a plurality of allele quantifications for a plurality of linked genomic loci.

8. The method of claim 7, wherein (i) a plurality of genotype likelihood sets for the target genomic locus is generated according to the plurality of allele quantifications for the plurality of linked genomic loci, wherein the genotype at the target genomic locus is generated based on the plurality of genotype likelihood sets.

9. (canceled)

10. The method of claim 8, wherein a composite genotype likelihood is generated for each genotype from the plurality of genotype likelihood sets, wherein the genotype at the target genomic locus is generated based on the composite genotype likelihoods.

11. (canceled)

12. The method of any one of claims 1 to 11, comprising generating a plurality of genotypes at a plurality of target genomic loci for the test sample, wherein each genotype in the plurality of genotypes is generated independently from the other genotypes in the plurality of genotypes, and identifying a subject based on the plurality of genotypes generated for the test sample.

13. (canceled)

14. (canceled)

15. The method of claim 1, wherein generating the genotype at the target genomic locus does not comprise generating a haplotype for two or more target genomic loci.

16. A method for generating a genotype for a target genomic locus for a test sample, comprising:
a) for a test sample comprising nucleic acid, obtaining sequence reads aligned to a reference genome;
b) for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group;
c) generating a haplotype pair likelihood set for the haplotype group according to i) the allele quantifications in (b), and ii) a probability of each haplotype pair; and
d) generating a genotype at the target genomic locus based on the haplotype pair likelihood set in (c).

17. The method of claim 16, wherein the haplotype pair likelihood set for the haplotype group is generated in (c) according to a Bayesian probability.

18. The method of claim 16, wherein the haplotype pair likelihood set for the haplotype group is generated in (c), given the allele quantifications in (b), according to i) a probability of the allele quantifications in (b) given each haplotype pair, and ii) a probability of each haplotype pair, wherein the probability in (i) is determined according to which genotype is most likely observed at each genomic locus across the haplotype group, given a particular haplotype pair.

19. (canceled)

20. The method of claim 18, further comprising calculating the probability of the allele quantifications in (b) at each at each genomic locus and generating a product across all genomic loci in the haplotype group.

21. The method of claim 16, wherein the probability of each haplotype pair in (c)(ii) is determined, in part, according to haplotype frequencies, wherein the probability of each haplotype pair in (c)(ii) is determined, in part, according to haplotype frequencies for (i) a target reference allele and the linked reference allele, (ii) a target reference allele and the linked alternative allele, (iii) a target alternative allele and the linked reference allele, and (iv) a target alternative allele and the linked alternative allele.

22. (canceled)

23. The method of claim 16, wherein (c) further comprises identifying the most probable haplotype pair from the haplotype pair likelihood set, wherein the genotype at the target genomic locus is generated in (d) according to the most probable haplotype pair.

24. (canceled)

25. The method of claim 16, wherein (c) further comprises aggregating the haplotype pair likelihoods across all haplotype pairs for the haplotype group, thereby generating aggregate likelihoods, wherein the genotype at the target genomic locus is generated in (d) according to the highest aggregate likelihood.

26. (canceled)

27. (canceled)

28. The method of claim 16, wherein (b) comprises, for a plurality of haplotype groups each comprising a target genomic locus and a plurality of linked genomic loci, quantifying a linked reference allele and quantifying a linked alternative allele for each linked genomic locus in each group according to the sequence reads generated in (a), thereby generating allele quantifications for each linked genomic locus for each group in the plurality of haplotype groups, wherein a plurality of haplotype pair likelihood sets are generated in (c) according to the allele quantifications for each linked genomic locus for each group in the plurality of haplotype groups, wherein a plurality of genotypes at a plurality of target genomic loci are generated in (d) based on the plurality of haplotype pair likelihood sets.

29. (canceled)

30. (canceled)

31. The method of claim 28, wherein each genotype in the plurality of genotypes is generated independently from the other genotypes in the plurality of genotypes.

32. The method of claim 28, further comprising identifying a subject based on the plurality of genotypes generated for the test sample.

33. The method of claim 16, wherein generating the genotype at the target genomic locus does not comprise generating a haplotype group comprising two or more target genomic loci.
